(19) United States
(12) Patent Application Publication
(10) Pub. No.: US 2026/0191749 A1
(43) Pub. Date: Jul. 9, 2026

(54) COMPOSITION AND PROCESS OF MAKING BIO-BASED UPCYCLED OILS IN FLOW REACTORS

(71) Applicant: RiKarbon, Inc., Newark, DE (US)

(72) Inventors: Basudeb Saha, Newark, DE (US); Arvind Nanduri, Wilmington, DE (US)

(73) Assignee: RiKarbon, Inc., Newark, DE (US)

(21) Appl. No.: 19/131,655

(22) PCT Filed: Jan. 12, 2023
(86) PCT No.: PCT/US2023/060535

§ 371 Date: May 21, 2025

Related U.S. Application Data
(60) Provisional application No. 63/384,831, filed on Nov. 23, 2022.

(51) Int. Cl. A61K 8/31; A61Q 17/04; A61Q 19/00; C07C 1/247; C07C 29/17; C07C 31/20; C07C 31/22; C07C 45/00; C07D 307/06; C07D 307/12; C08G 65/26; C10M 105/14

(52) U.S. Cl. CPC A61K 8/31; A61Q 17/04; A61Q 19/007; C07C 1/247; C07C 29/172; C07C 31/20; C07C 31/22; C07C 45/002; C07D 307/06; C07D 307/12; C08G 65/2609; C10M 105/14; C10M 2207/0225

(57) ABSTRACT

Composition and efficient manufacturing processes for making up to 100% bio-based and upcycled renewable oils in batch and flow reactors using raw-materials, sourced from non-food biomass and/or inedible oil seeds/natural oils, is described. The improved manufacturing processes with concurrent removal of process generated water (for RKBA-n) and shorter reaction time and hence reduced overall energy consumption of the manufacturing of products are exemplified. Composition of new bio-based oils containing terminal hydroxyl groups is described.

### CROSS REFERENCE TO RELATED APPLICATIONS

This application is based on U.S. Provisional Patent Application No. 63/384,831, filed on Nov. 23, 2022. The priority of the foregoing application is hereby claimed, and its disclosures incorporated herein by reference in its entirety.

### BACKGROUND

Different conventional oils are used as emollients and base-oils in cosmetic and lubricant products formulations. Emollient, also referred to as cosmetic oil, is used in various cosmetics for easing solubilization of emulsifiers in water-oil emulsions and providing aesthetic feelings as well as desired efficacy (spreadability, and dry, shiny, silky and talc feelings). The chemistry of emollient, and their application in cosmetics formulation for various desired properties have evolved over the years. One of the noticeable changes is the widespread use of cyclic silicones, also referred to as cyclosiloxanes (trade names D4, D5, D6). While these cyclic compounds, with different degree of volatility profile, are serving the most cosmetic products formulation needs, they have been highly scrutinized in the recent years because of their safety and bio-accumulation concerns. Since January 2020, the use of these cyclic silicones has been restricted in most wash-off cosmetics. In addition, generation X and generation Z consumers' preference for natural, non-silicon clean beauty products have increased over the last decade. Recent consumer review statistics indicates that an average consumer of all age groups avoid silicones in cosmetic products.[1] As a result, alternative non-silicon ingredients such as linear/branched hydrocarbons (isododecane, combination of different hydrocarbons mixture,[2] squalane/isosqualane[3], pentadecane, hexadecane[4]) and esters (Neopentylglycol diheptanoate[5], PEG/PPG-8/3 Laurate[6], combination of esters (PPG-3 isostearyl methyl ether, PPG-3 benzyl ether myristate, PPG-3 benzyl ether ethylhexanoate, isoamyl cocoate, and diethylhexyl carbonate) [7] and others) have been looked into by cosmetics manufacturers and formulators. However, most of these alternative ingredients are not derived from bio-based raw materials, especially non-food, and sustainable, typically waste, raw materials. Importantly, business insider analysis (based on customer interviews) indicated most alternative ingredients do not perform as good as cyclic silicones. Conventional petroleum-based mineral and synthetic oils are used as base oils in the formulation of end-use lubricants. Base oils constitute the major component (75-98 wt %) in lubricants, followed by various additives, e.g., antioxidants, pour point depressants, viscosity index improvers, corrosion inhibitors, anti-wear agents and others. The additives are carefully formulated with base oils to achieve desired specifications for automotive, industrial, metal-working, marine, mining, locomotive, chainsaw, golf cart, hydraulic fluids, grease and other consumer and industrial applications. Some of these applications have high risk of environmental exposure of petroleum-derived lubricants, which pose a significant monetary and regulatory concern to the users, local, State and Federal Government, regulatory bodies, and environmental advocates. Environmental regulations (e.g., REACH in Europe and VGP in USA) and the growing demand for sustainable lubricants to mitigate regulatory challenges and improve energy efficiency, fuel economy and carbon footprint have driven development of bio-based base oils.

### SUMMARY

In general, this invention relates to the synthesis of a range of bio-based upcycled oils. These oils are branched hydrocarbons and have carbon numbers in the range of 11 to 33. All products contain up to 100% bio-based carbon and were synthesized from commercially precured biomass and/or seed oils/natural oils derived raw materials. Up to 100% carbon of these raw materials comes from sustainably sourced, typically waste, feedstocks. An improved manufacturing process to make oils and their key physical characteristics and specifications are described.

### BRIEF DESCRIPTION OF THE FIGURES

The accompanying drawings, which are incorporated in and constitute a part of this specification, illustrate embodiments, and together with the general description given above, and the detailed description of the embodiments given below, serve to explain the principles of the present disclosure.

FIG. 1 is a drawing of molecular structures of bio-based upcycled oils.

FIG. 2 is a photograph of an embodiment of an oil-heated tubular reactor system.

FIG. 3 is a schematic of an embodiment of a tubular reactor system.

FIG. 4 is a schematic of embodiments of catalyst packing for tubular reactors.

FIG. 5 is a reaction scheme of hydrodeoxygenation.

FIG. 6 is a gas chromatograph (GC) chromatogram profile of RKunsat-17.

FIG. 7 is a gas chromatograph (GC) chromatogram profile of RKunsat-22.

FIG. 8 are graphs of yields of condensation products as a function of time.

FIG. 9 are graphs of yields under different reaction conditions.

FIG. 10 is a total ion chromatograph (TIC) gas chromatograph (GC) chromatogram of Rkunsat-28/33.

FIG. 11 is a total ion chromatograph (TIC) gas chromatograph (GC) chromatogram of Rkunsat-28/33.

FIG. 12 a total ion chromatograph (TIC) gas chromatograph (GC) chromatogram of RKunsat-14.

FIG. 13 is a graph of the effect of liquid weight hourly space velocity (LWHSV) and temperature on RKunsat-22 conversion.

FIG. 14 is a gas chromatograph (GC) chromatogram of RKunsat-22.

FIG. 15 is a gas chromatograph (GC) chromatogram of an HDO product from the conversion of hydrogenated intermediate of RKunsat-22 using a tubular reactor.

FIG. 16 is a gas chromatograph (GC) chromatogram of furan ring hydrogenated intermated of RKunsat-17.

FIG. 17 is a graph of the effect of flowrate and furnace temperature on the reaction exotherm.

FIG. 18 is a gas chromatograph (GC) chromatogram of RKBA-17 product collected from the tubular reactor.

FIG. 19 is a gas chromatograph (GC) chromatogram of the hydrogenated product of RKunsat-28/33.

FIG. 20 is a drawing of molecular structures of different species identified in the hydrogenated intermediate product of RKunsat-28/33.

FIG. 21 shows the IUPAC names of different species identified in the hydrogenated intermediate product of RKunsat-28/33.

FIG. 22 is a gas chromatograph (GC) chromatogram of RKunsat-28/33.

FIG. 23 is a gas chromatograph (GC) chromatogram of RKBA-28/33.

FIG. 24 is a gas chromatograph (GC) chromatogram of RKBA-28/33.

FIG. 25 is a gas chromatograph (GC) chromatogram of RKBA-14.

### DETAILED DESCRIPTION

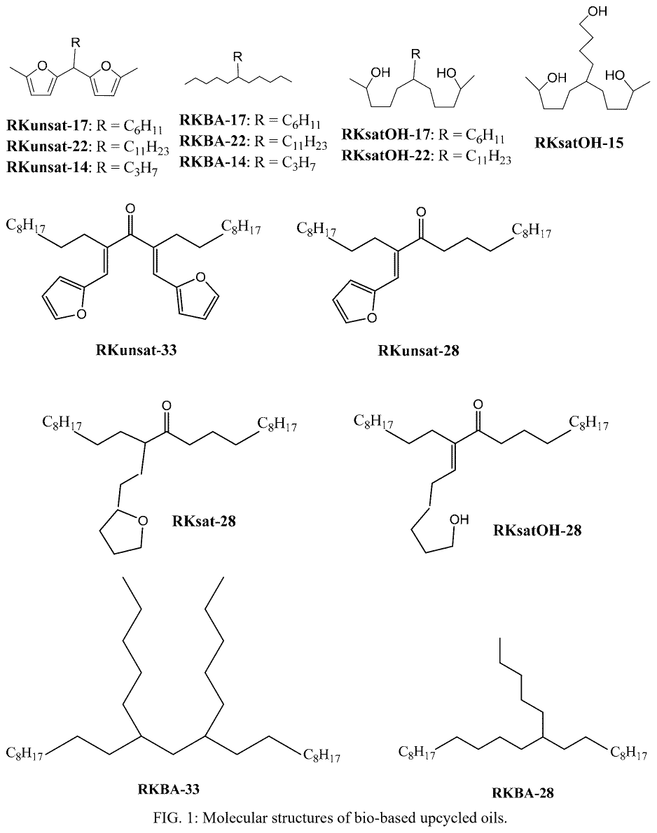

The present invention is related to the improved manufacturing process of bio-based renewable branched hydrocarbon oils in flow and batch reactors. General molecular formulae of these branched hydrocarbon oils and their precursors containing furan rings, are RKBA-n and RKunsat-n respectively, where n represents total number carbon atoms in the molecular structures of compounds. Specifically, it discloses manufacturing processes of compounds of formulae RKBA-17, RKBA-22, RKBA-14, RKBA-28/33 (FIG. 1). Their precursors RKunsat-14, RKunsat-17, RKunsat-22, RKunsat-28/33 were synthesized and scaled up. This invention also discloses new composition of bio-oils containing two and more terminal hydroxyl groups such as RKsatOH-17, RKsatOH-22, RKsatOH-14, RKsatOH-28 and RKsatOH-15 (FIG. 1).

Example of these compounds include:

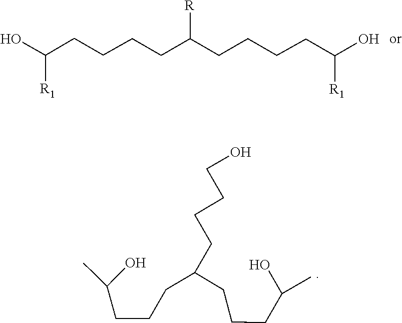

Wherein R is methyl, npropyl, nhexyl, nbutyl, n-pentyl, or nundecyl and R₁ is independently selected from H or an alkyl chain of carbon number 1-7. Other compounds include:

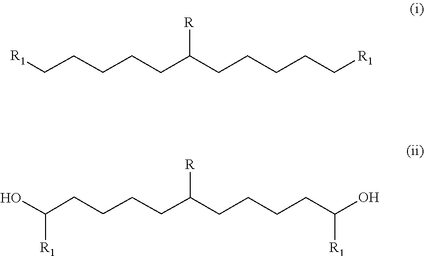

wherein R is methyl, npropyl, nhexyl, nbutyl, n-pentyl, or nundecyl and R₁ is independently selected from H or an alkyl chain of carbon number 1-7. Examples of other compounds include:

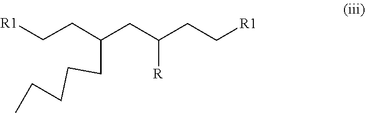

wherein R1 is independently selected from alkyl groups of carbon number 2 to 14, R is independently selected from H or —(CH2) 4 group. Examples of other compounds include:

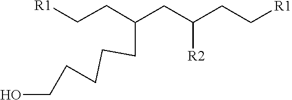

wherein R1 is independently selected from alkyl groups of carbon number 2 to 14, and R2 is H or —(CH2)4.

In some embodiments, a composition comprises the any of the above compounds with at least one branched carbon chain. The composition comprises a bio-based content in the range of 20 to 100%, according to ASTM-D6866 and hydroxyl content of greater than 20 mg KOH/per gram, according to ASTM 4274-99 method.

The syntheses involved two-steps catalytic reactions of commercially procured bio-based raw materials, derived either from non-food biomass and/or inedible oil seeds/natural oils, followed by distillation and purification. The catalysts were prepared in-house or procured from commercial sources.

A synthetic method may be used to make compounds having the following structures:

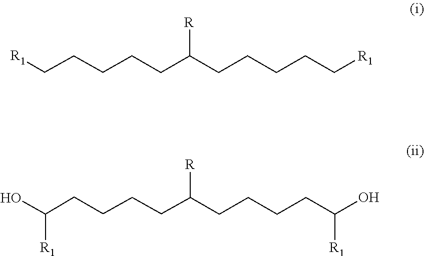

wherein R is methyl, npropyl, nhexyl, nbutyl, n-pentyl, or nundecyl and R1 is independently selected from H or an alkyl chain of carbon number 1-7. In some embodiments, the method comprises of the steps of: (a) an acid catalyzed condensation reaction of a first component comprising one of more of a 2-alkylfuran wherein the alkyl group is independently selected from carbon number of 1 to 7, with a second component comprising an aldehyde having the formula of O═CH—R wherein R is methyl, npropyl, nhexyl, nbutyl, n-pentyl, or nundecyl, to produce a condensation compound. (b) Optionally, the condensation product may undergo selective hydrogenation in the presence of a hydrogenation catalyst to obtain a hydrogenated saturated or ring-opened hydroxyl compound. (c) Selective hydrodeoxygenation of the condensation compound or the hydrogenated saturated or ring-opened hydroxyl compound, in the presence of a hydrodeoxygenation catalyst results in a branched alkane compound.

A synthetic method may be used to make compounds having the structure:

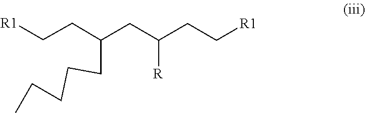

wherein R1 is independently selected from alkyl groups of carbon number 2 to 14, R is independently selected from H or —(CH2) 4 group. In some embodiments, the method comprises of the steps of: (a) a base catalyzed condensation reaction of a third component with furfural. The third component comprises one of more of a ketone of molecular formulae (R1) (R1)C═O wherein R1 is independently selected from alkyl groups of carbon number from 2 to 14. The base catalyzed condensation products compounds of structure (i):

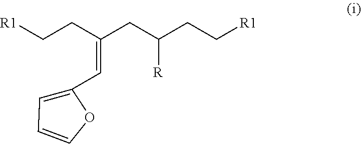

wherein R is H or a furan ring. (b) Optionally, the base catalyzed condensation product may undergo selective hydrogenation in the presence of a hydrogenation catalyst to obtain furan ring saturated intermediates or ring-opened hydroxyl product to produce compounds of structure (ii):

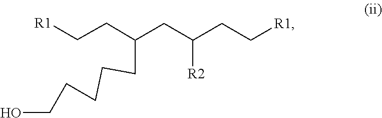

wherein and R2 is H or —(CH2) 3CH₂OH group. (c) Selective hydrodeoxygenation of the condensation compound of structure (i) or the hydrogenated saturated or ring-opened hydroxyl intermediate of structure (ii) in the presence of a hydrodeoxygenation catalyst results in a branched alkane compound of structure (iii).

The first step involves catalytic condensation reaction of biomass and other bio-sourced feedstock derived raw materials in a batch reactor (glass or metal) to produce a condensation compound (RKunsat-14, RKunsat-17, RKunsat-22, RKunsat-28/33) for bio-based branched hydrocarbon oils. Examples of the catalytic condensation reaction include an acid catalyzed hydroxyalkylation/alkylation condensation reaction and a base catalyzed aldol condensation reaction.

In some embodiments, the catalytic condensation is an acid catalyzed hydroxyalkylation/alkylation condensation reaction of a first component and a second component. The hydroxyalkylation/alkylation catalyzed condensation reaction uses an acidic catalyst. Examples of acidic catalysts include, but are not limited to organic or inorganic liquid acids and solid Bronsted acids such as, but not limited to, acidic resins, fluorinated resins, zeolites, phosphoric acid, phosphorous silica, orthophosphoric acid, HCl, H₂SO₄, methanesulfonic acid, p-tolunesulfonic acid etc. In some embodiments, the acidic catalysts is selected from catalytic materials containing Bronsted acid sites of which the catalysts containing weakly Bronsted acid sites, e.g., phosphorous silica. In-house synthesized and/or commercial procured solid acid catalysts were used for the syntheses of condensation compounds: RKunsat-14, RKunsat-17, RKunsat-22. Phosphorous silica may be prepared by wetness impregnation of an aqueous solution of o-phosphoric acid on mesoporous silica support

In some embodiments, a first component comprises one of more of a 2-alkylfuran wherein the alkyl group is independently selected from carbon number of 1 to 7, or combinations thereof. In some embodiments, the 2-alkylfuran is selected from 2-methylfuranfuran, 2-ethylfuran, and 2-propylfuran. The first component may be in a pure form or contain biogenic impurities, such as those formed in the process of making the 2-alkylfuran.

In some embodiments, a second component comprises an aldehyde having the formula of O═CH—R wherein R is methyl, npropyl, nhexyl, nbutyl, n-pentyl, or nundecyl. In some embodiments, the aldehyde is selected from npropyl, nhexyl, and nundecyl. The second component may be in a pure form or contain process impurities. The aldehydes can be bio-sourced and/or fossil-based in pure form or contain process impurities. In some embodiments, the aldehydes are from bio-sourced materials and may contain biogenic impurities.

In some embodiments, the catalytic aldol condensation is a base catalyzed condensation reaction of a third component and furfural. The base catalyzed aldol condensation reaction uses a basic catalyst. Examples of basic catalysts include, but are not limited to homogeneous and heterogeneous inorganic bases such as, but not limited to, CaO, MgO, Al—Mg hydrotalcites of different particle sizes and base density, NaOH, KOH, Ca(OH)₂, Mg(OH)₂ etc. In some embodiments, the base catalyst is selected from catalysts with higher basic sites, e.g., NaOH, KOH. In some embodiments, the base catalysts are selected from catalysts that contain high base sites and heterogeneous, e.g. CaO. In some embodiments, CaO catalyst is used for the synthesis of aldol condensation compounds: RKunsat-28/33. In some embodiments, NaOH catalysts is used to synthesize condensation compounds: RKunsat-28/33.

In some embodiments, a third component comprising one of more of a ketone of molecular formulae (R1)(R1)C═O wherein R1 is independently selected for alkyl group of carbon number from 2 to 14. In some embodiments, the ketones are in pure form. In some embodiments, the ketones are bio-sourced ketones containing alkyl groups of carbon number between 5 to 12.

In some embodiments, bio-based 2-alkylfurans and furfural refer to such materials that are produced from biomass via biomass saccharification, dehydration of biomass sugars to furfural and hydrogenolysis of furfural to 2-methylfuran. In some embodiments, 2-alkylfurans containing alkyl groups of carbon numbers 2 or greater than 2 are synthesized via catalytic decarbonylating of bio-based furfural to furan, acylation of furan with bio-based or fossil-based carboxylic acids and/or anhydrides to acylated compounds, followed by selective hydrogenation of acylated compounds to 2-alkylfurans. In some embodiments, bio-sourced raw materials refer to raw materials that are derived from lauric acid (obtain from coconut oil or palm kernel oil), castor seeds, and other bio-based feedstock which may be biomass or bio-based carbon.

Examples of the catalysts for the selective hydrogenation include, but are not limited to: Ni, Pd, Pt, Ru, and other hydrogenation metals supported on a support material such as activated carbon, porous carbon, polymeric hybrid material, weakly acidic materials etc., and mixed Ni catalyst in the presence of NiO, Al2O3, SiO₂, Cr2O3, ZrO2, or Kieselguhr. Selective hydrogenation refers to the reactions that can selectively hydrogenate the furan rings to produce RKsat compounds or can selectively hydrogenate furan rings and undergo ring opening to produce RKsat-OH and/or a mixture of RKsat and RKsat-OH. In some embodiments, the hydrogenation catalyst is selected from one or more of powder and/or pelleted form of Ni/C, Raney Ni, Pd/C, Ru/C, Ni/SiO2, Pd/SiO2, mixed Ni-based oxide catalysts, Ni/Al2O3, and Ni/SiO2—Al2O3. In some embodiments, the hydrogenation catalyst is selected from Raney Ni, Pd/SiO2 Ni/C and mixed Ni and Ni-oxides along with other oxides such as Al2O3, SiO2, Cr2O3, ZrO2, and Kieselguhr. In some embodiments, the selective hydrogenation is conducted in a metallic pressure reactor or in a flow reactor under H₂ pressure ranging from 1 bar to 45 bar and temperature in the range of 40° C. to 200° C.

The second step involves catalytic hydrodeoxygenation (HDO) of the condensation compounds to the corresponding branched hydrocarbon compounds. In some embodiments, the HDO reaction is done in high pressure metal reactors in batch operation using a non-polar solvent. This reaction produced 2 moles of water per mole of substrates. It was observed during scale up reaction that accumulated water in the reactor slows down the reaction and then completely stopped the reaction, likely because of coverage of the catalyst surface by water. Removal of the water with the solvent under N₂ purge, separation of the water from the solvent, and then returning the solvent to the reactor allowed the reaction to resume.

For example, a HDO reaction of 350 g RKunsat-22RKunsat-22 as a precursor with 12 g Ni62/15P catalyst in a 1 gallon reactor was initiated. First, the catalyst was pre-activated in cyclohexane at 160° C. under 5 bar H₂ for 2 h. Then the precursor was fed to the reactor and the reaction started at 240° C. under 45 bar H₂. The reaction product was collected as a function of time. It showed that after 3.5 h of reaction, H₂ consumption was very slow because of water accumulation in the reaction mass. The reaction solution was cooled down to 70° C., solvent along with accumulated water by-product was stripped off, new solvent was fed in and the reaction resumed again at 240° C. under 45 bar H₂. Using new solvent, the reaction initiated again and completed. It is believed that accumulated water slowed down the reaction. When the reaction was scaled up in a 30 gallon pressure reactor, the reaction was stopped three times to vacuum strip water.

In some embodiments, the HDO reaction can be conducted without using a solvent. Vacuum stripping of accumulated water from the reactor was performed during the reaction by cooling down the reactor intermittently. For example, a HDO reaction of 50 kg of RKunsat-22 was conducted in a 30 gallon reactor using 2 kg of Ni62/15 catalyst. The catalyst activation was done at 160° C. for 2 hours under 70 psi H₂. Then the temperature was increased to 220-240° C. to continue the HDO reaction under 600 psi H₂ pressure for 3 hours. The reactor was cooled down to 120° C. to remove the water co-product using vacuum under N₂. Then the reaction resumed at 240° C. under 600 psi pressure for additional 3 hours followed by water removal. A total of 6.4 kg water was removed in this step. HDO step resumed again for additional 3 hours when H₂ consumption rate was very slow. The reactor was cooled down, H₂ was released, and the product was collected by vacuum filtration of the catalyst from the reaction mass and finally the colorless and odorless RKBA-22 product was distilled to separate any light alkanes.

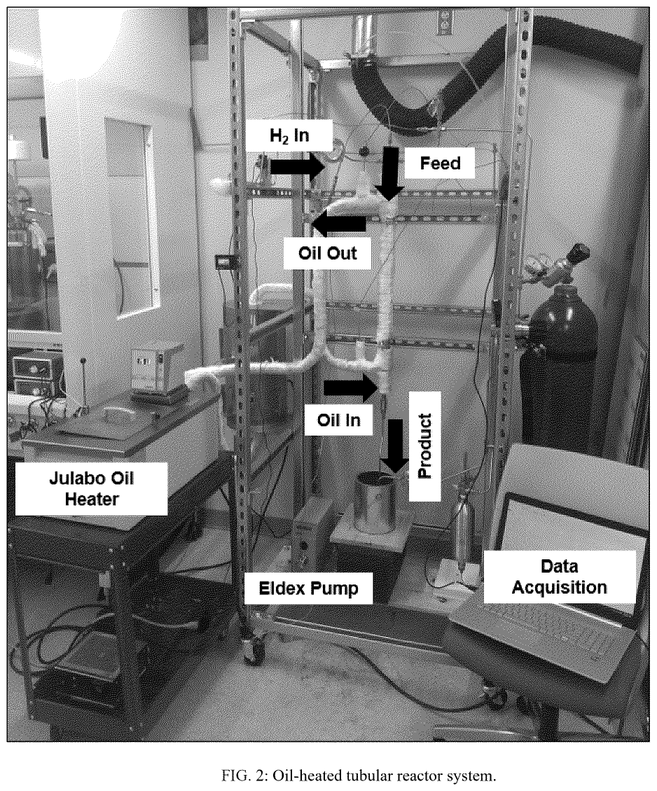

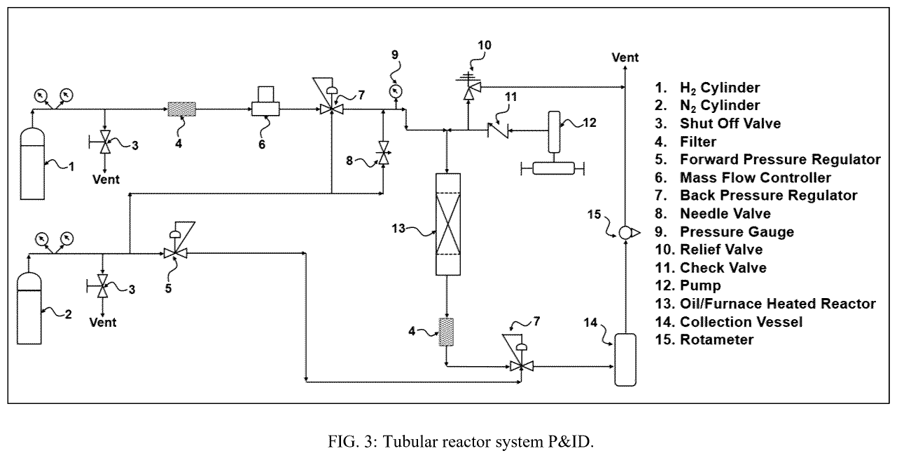

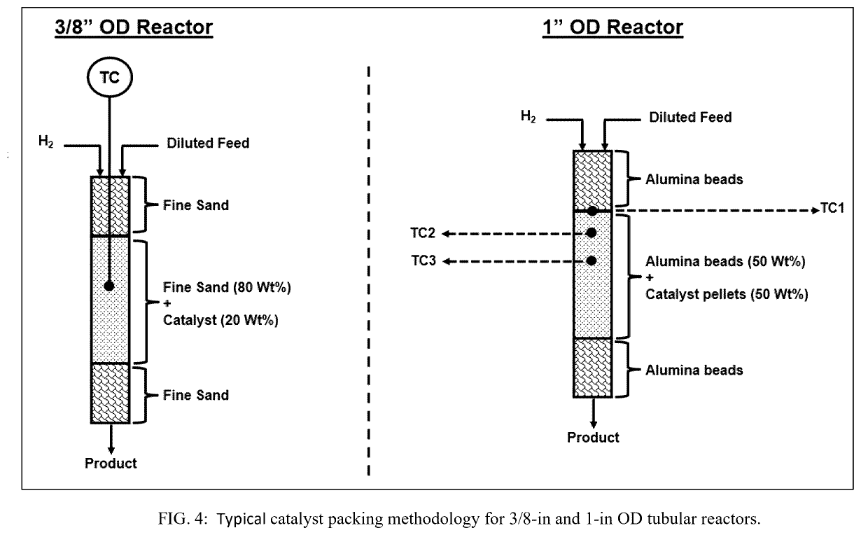

In some embodiments, a flow reactor is used to conduct the HDO reaction to enable water co-product to continuously flow down the catalyst bed as it is produced. In some embodiments, a tubular reactor system comprised an in-house constructed 2 ft long 1.0″ OD SS tube with ~11″ of active catalyst packing heated by a tubular furnace. The reactor was equipped with a furnace, mass flow controller, temperature and pressure controllers, pressure relieve valves, and online data collection protocol. The reactor system and the corresponding P&ID are shown in FIGS. 2 and 3. The reactor temperature, furnace set temperature and shell-side oil temperature (if oil bath was used for heating the catalyst bed and to transfer heat from the catalyst bed) were recorded using a DATAQ DI-2008 data logging system. The experiments were performed using a 12″ and 18″ bed length PRICAT Ni 60/15T pellet (name of the pelleted form of Ni62/15P catalyst with the same oxide composition) of sizes 250-700 microns packing diluted with similar size inert particles. In some embodiments, cylindrical pellet of size 4 mm×5 mm may be used. An example packing methodology is shown in FIG. 4.

In some embodiments, RKunsat-n is converted to RKBA-n in this reactor under adiabatic operation conditions at 120° C., ~600 psi H₂, 3 ml/min flow rate of neat feed (no solvent). The reactions at 12 g/min of H₂ flow resulted in a significant exotherm of 260° C. in magnitude at a set up temperature of 120° C. The heat formation largely occurred in the beginning of the reaction when furan rings of RKunsat-22 are hydrogenated. In some embodiments, the HDO reaction is performed stepwise. First, furan rings hydrogenation of the feed, e.g. RKunsat-22, RKunsat-17, RKunsat-14, and RKunsat-28/33, was conducted in a batch reactor to produce hydrogenated feed. This step does not produce water co-product. The hydrogenated feed is then pumped through the catalyst bed of the tubular reactor to produce the final deoxygenated products, e.g. RKBA-22, RKBA-17, RKBA-14, and RKBA-28/33 containing other C—C cleavage species in RKBA28/33. The deoxygenation reaction step involved ring-opening of the hydrogenated feed to hydroxyl intermediates, dehydration of hydroxyl intermediates to olefinic species and the hydrogenation of olefinic species to branched alkanes (FIG. 5). Water is formed as a co-product in the deoxygenation step which flows through the catalyst bed along with the product and did not inhibit the reaction as it was observed in the batch process. The product was collected in a collection vessel in which higher density water phase separated from the lower density alkane products. The final product was collected from the top phase. The yield, purity and selectivity of each product were determined using chromatography and mass spectrometry techniques.

Examples of the hydrodeoxygenation catalyst include, but are not limited to a metal selected from a hydrogenation metal or a base metal, and an acid site selected from a Lewis acid or a Bronsted acid. In some embodiments, the metal-acid catalyst for the hydrodeoxygenation reaction can be a bifunctional catalyst in powder and pellet forms such as Ni/ZSM5, Ni/zeolite, Ni/silica, Ni/Al2O3, Pd/ZSM5, Pd/zeolite, Pd/silica, Pd/Al2O3, a physically mixed oxide catalysts consisting of at least one metal oxide such as NiO and at least one acidic oxide such as alumina, silica, or a supported metal-metal oxide catalyst of a general formula MIMO wherein M¹=Ir, Ru, Ni, Co, Pd, or Rh and M=Re, Mo, W, Nb, Mn, V, Ce, Cr, Zn, Co, Y, or Al. In some embodiments, the catalyst is selected from Ni/ZSM5, Ni/zeolite and a physically mixed oxide catalysts of at least one metal oxide such as NiO and at least one acidic oxide such as alumina, silica.

In some embodiments, the hydrodeoxygenation reactions are conducted in the flow reactor of varying length of the catalyst bed and temperature ranging from 150° C. to 300° C., such as 180° C. to 250° C., under varying flow rates of feed and H₂ pressure ranging from 80 psi to 800 psi. In some embodiments, the pressure ranges from 200 psi to 600 psi. In some embodiments, the hydrodeoxygenation reaction is conducted in the flow reactor with a catalyst bed length ranging from 8″ to 240″, such as from 8″ to 18″, from 12″ to 240″, from 8″ to 12″, from 8″ to 12″, from 12″ to 24″, from 24″ to 36″, from 36″ to 48″, from 48″ to 60″, from 60″ to 72″, from 72″ to 90″, from 90″ to 110″, from 110″ to 130″, from 130″ to 150″, from 150″ to 175″, from 175″ to 200″ from 200″ to 225″, and from 225″ to 240″. In some embodiments, the flow reactor's outer diameter from ⅜″ to 2″, such as from 1″ to 4″ using a feed flow rate from 0.1 mL/min to 2 mL/min, such as from 1 mL/min to 270 mL/min. In some embodiments, the water by-product is separated in a collection vessel from the product of structure RKBA-n.

In some embodiments, a composition comprises a compound made by any of the above methods wherein the composition has a bio-based carbon content in the range of 10% to 100%, according to ASTM-D6866.

In some embodiments, a cosmetic product formulation may comprise 0.1-99% by weight of one or more compound described above as an emollient and an effective amount of one or more cosmetic additives. In some embodiments, the cosmetic product formulation further comprising a compound of structure of:

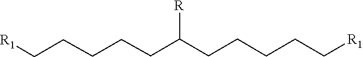

wherein R is methyl, npropyl, nhexyl, nbutyl, n-pentyl, or nundecyl and R1 is independently selected from H or an alkyl chain of carbon number 1-7; or

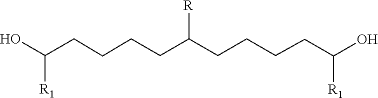

wherein R is methyl, npropyl, nhexyl, nbutyl, n-pentyl, or nundecyl and R1 is independently selected from H or an alkyl chain of carbon number 1-7. In some embodiments, the cosmetic formulation further comprises a cosmetic oil.

In some embodiments, A cosmetic product composition comprises (i) emollients comprising one or more compound described above, (ii) one or more additives selected from the group of pigment, fragrance, emulsifier, wetting agent, thickener, emollient, rheology modifier, viscosity modifier, gelling agent, antiperspirant agent, deodorant active, fatty acid salt, film former, anti-oxidant, humectant, opacifier, monohydric alcohol, polyhydric alcohol, fatty alcohol, preservative, pH modifier, a moisturizer, skin conditioner, stabilizing agent, proteins, skin lightening agents, topical exfoliants, antioxidants, retinoids, refractive index enhancer, photo-stability enhancer, SPF improver, UV blocker, antibiotic agents, antiseptic agents, antifungal agents, corticosteroid agents, anti-acne agents, and water.

In some embodiments, a lubricant product formulation comprising 0.1-99% by weight of one or more compound described above and an effective amount of one or more lubricant additives. In some embodiments, the lubricant product formulation further comprising a compound of structure of:

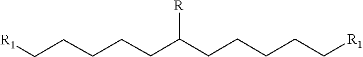

wherein R is methyl, npropyl, nhexyl, nbutyl, n-pentyl, or nundecyl and R₁ is independently selected from H or an alkyl chain of carbon number 1-7;

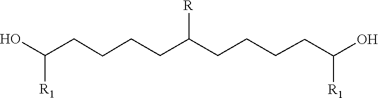

wherein R is methyl, npropyl, nhexyl, nbutyl, n-pentyl, or nundecyl and R₁ is independently selected from H or an alkyl chain of carbon number 1-7; or

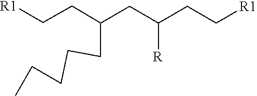

wherein R1 is independently selected from alkyl groups of carbon number 2 to 14, R is independently selected from H or —(CH2) 4 group

In some embodiments, a lubricant composition comprises (i) a base oil comprising one or more compound described above, and (ii) a lubricant additive selected from thickener, viscosity modifier, base oils, viscosity index improver, pour point depressant, anti-wearing agent, corrosion inhibitor, anti-oxidant, extreme pressure additive, detergents, and anti-foaming agent.

The various RKsatOH-n compounds may be used as monomers, crosslinkers, and chain extenders to form polymers. The RKunsat-n compounds and saturated furan compounds may be used as precursors for making RKsatOH-m monomers in the polymerization. Examples of polymers made with these alcohol monomers includes polyurethanes, polyesters, alkyd resins, polyethers, etc.

The properties of bio-based carbon content, stability, and toxicity assessment of the synthesized oils were evaluated.

### EXAMPLES

The composition of RKsatOH product and the process of making of the aforementioned products are exemplified below.

### Example 1. Preparation of RKunsat-17

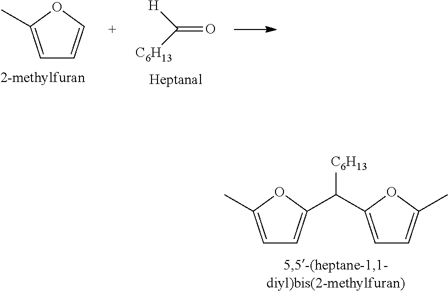

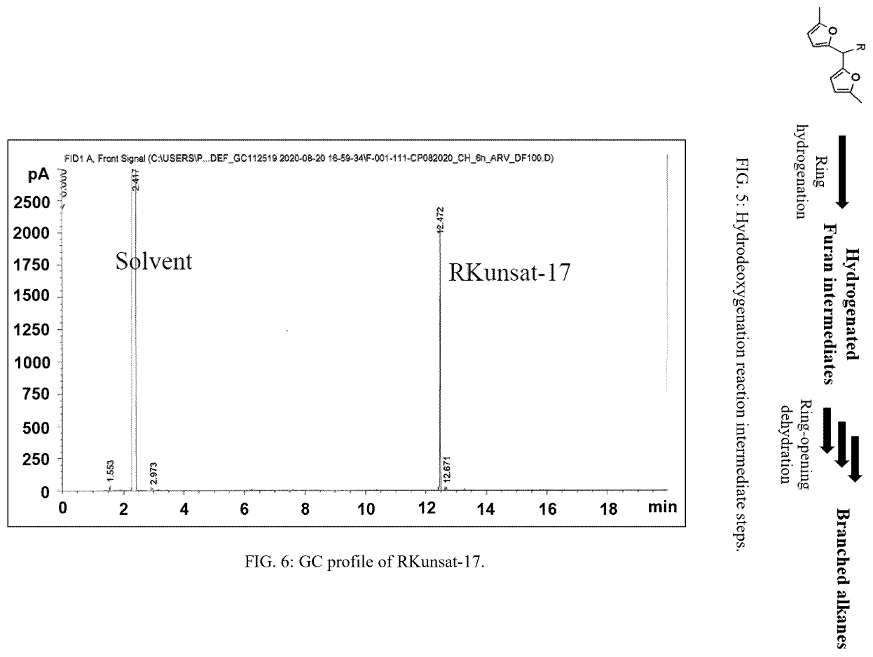

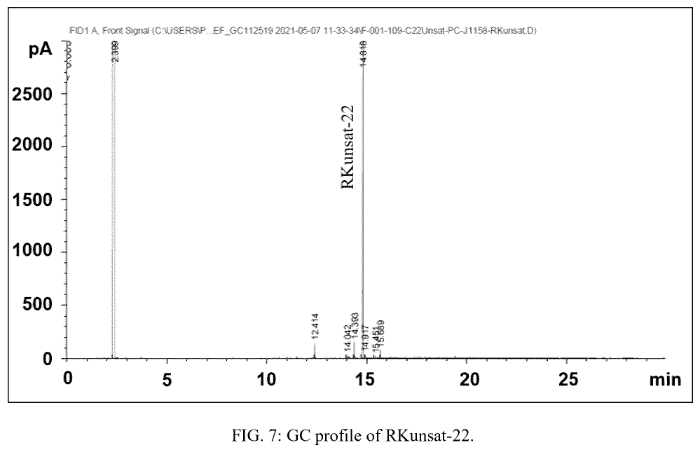

RKunsat-17 was prepared by catalytic condensation between commercially procured biomass and/or castor seed derived heptanal and 2-methylfuran (2MF) in the temperature range of 60° C. to 80° C. In-house prepared P—SiO2 catalyst (P=phosphorous) with 10 wt % phosphoric acid loading (3.2% P) or commercially procured fluorinated resin catalyst, Aquivion PW98, was used as an acid catalyst for the reaction. Molar ratio of 2MF to heptanal was 3 to push the reaction equilibrium towards completion and ensure the product does not contain any residual heptanal to avoid the separation complexity. The reaction temperature was sequentially increased—60° C. for first 30 min, 70° C. for next 30 min and 80° C. for the rest of the reaction time—to reduce polymerization of 2MF on the catalysts surface in the beginning of the reaction. A Dean-Stark apparatus was used for the separation of water-co-product during the reaction. Table 1 summarizes representative results using both catalysts. It shows that P—SiO2 catalyst is more effective to achieve optimal yields RKunsat-17 with higher selectivity. After the reaction, the product was vacuum distilled in the temperature range of 50° C. to 70° C. remove remaining 2MF and water co-product. Purity of RKunsat-17 was over 99% on the basis of GC analysis (FIG. 6). The color of the product was light greenish orange.

TABLE 1
Production of RKunsat-17 on different scales under various reaction conditions.

| | Reaction | | Yield of | Conv of |
|---|---|---|---|---|
| Entry # | scale | Catalyst & reaction conditions | RKunsat-17 | heptanal |
| 1 | 650 g | P-SiO2; 60° C. for 0.5 h, 70° C. for 0.5 h | 98% | 98% |
| | | and 80° C. for 5 h | | |
| 2 | 2.5 kg | P-SiO2; 60° C. for 0.5 h, 70° C. for 0.5 h | 97% | 97% |
| | | and 80° C. for 6 h | | |
| 3 | 200 g | P-SiO2; 60° C. for 0.5 h, 70° C. for 0.5 h | >99% | >99% |
| | | and 80° C. for 4 h | | |
| 4 | 130 g | P-SiO2; 60° C. for 0.5 h, 70° C. for 0.5 h | >99% | >99% |
| | | and 80° C. for 4 h | | |
| 5 | 130 g | Aquivian PW98; 60° C. for 0.5 h, 70° C. | 98% | 98% |
| | | for 0.5 h and 80° C. for 5 h | | |
| 6 | 130 g | Aquivian PW98; 60 ° C. for 2 h, and 80° C. | 98% | >98% |
| | | for 7 h | | |

### Example 2. Preparation of RKunsat-22

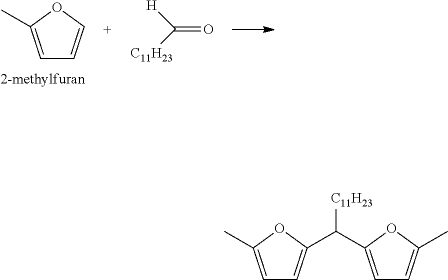

RKunsat-22 was prepared by catalytic condensation between commercially procured biomass derived 2MF and fatty acid and/or palm kernel derived dodecanal in the temperature range of 60° C. to 90° C. The experimental and product purification methodologies were similar to those of RKunsat-17 product. The staged heating enabled better yield and product selectivity when compared with the results disclosed in prior art. Table 2 summarizes representative results. It shows that P—SiO₂ catalyst is more effective to achieve optimal yields of RKunsat-22 with higher selectivity. The process was scaled up to produce up to 70 kg RKunsat-22. Because water was formed as a co-product in the process, its accumulation in the reactor in the larger scale reaction caused slow down the reaction at the later stage. Thus, temperature was increased to 98 C to evaporate water to the Dean-Stark set up. In case of large scale reaction, the catalyst was vacuum filtered using a 0.5 micro filter. Any residual 2MF and water in the product was distilled off. Purity of RKunsat-22 after distillation was up to >99% on the basis of GC analysis (FIG. 7). The color of the product was light orange.

TABLE 2
Production of RKunsat-22 on different scales under various reaction conditions.

| | Reaction | | Yield of | Conv of |
|---|---|---|---|---|
| Entry # | scale | Catalyst & reaction conditions | RKunsat-22 | dodecanal |
| 1 | 33 | P-SiO2; 50° C. for 0.5 h, 60° C. for 0.5 h, | 97% | 98% |
| | kilogram | 70° C. for 3 h and 82° C. for 2 h | | |
| 2 | 70 | P-SiO2; 60° C. for 0.5 h, 65° C. for 0.5 h, | 97% | 98% |
| | kilogram | 70° C. for 3 h and 98° C. for 2 h | | |
| 3 | 1.4 | P-SiO2; 60° C. for 0.5 h, 70° C. for 0.5 h, | 96% | 98% |
| | kilogram | 80° C. for 7 h | | |
| 2 | 175 g | P-SiO2; 60° C. for 0.5 h, 70° C. for 0.5 h | >99% | >99% |
| | | and 80° C. for 5.5 h | | |
| 3 | 175 g | Aquivion PW98; 60° C. for 0.5 h, 70° C. | 94% | 95% |
| | | or 0.5 h and 80° C. for 8.5 h | | |

### Example 3. Preparation of RKunsat-28/33 (a mixture of RKunsat-28 (11E)-11-(furan-2-ylmethylidene)tricosan-12-one)) and RKunsat-33 (((11E,13E)-11,13-bis(furan-2-vlmethylidene)tricosan-12-one))

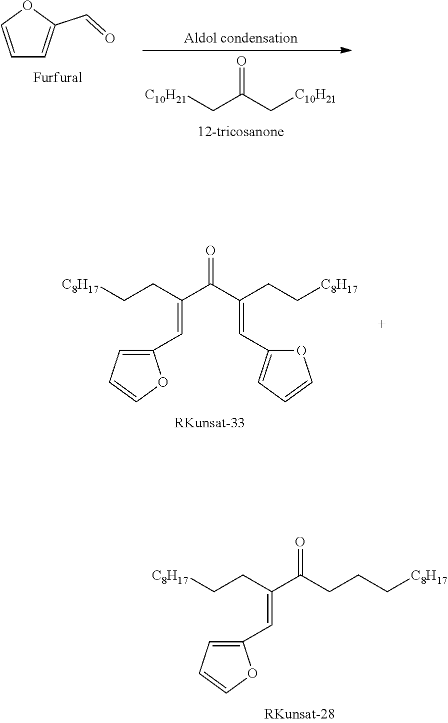

RKunsat-28/33, which contains RKunsat-28 as the major component and a small amount of RKunsat-33 was prepared by base catalyzed aldol condensation reaction of commercially procured biomass derived furfural (FF) and 12-trocosanone (Tr). Commercial CaO of particle size of 850 micron and 10 micron was used as a base catalyst for the first time in our synthesis. In prior report, NaOH was used as the base catalyst for aldol condensation reaction.[8]

Initial small scale reactions in glass vials or in autoclaves were performed using CaO of 10 micron size (Table 3). The reaction time, temperature, molar ratios of FF and Tr and solvents were varied. The solvent variation included methanol, ethanol, and isopropanol. It was found that methanol achieved the best performance.

TABLE 3
Production of RKunsat-28/33 on different scale using CaO (10 micron) catalyst.

| | | | FF:TRI | Yield of product % | | Yield of product % | |
|---|---|---|---|---|---|---|---|
| | Temp | CaO | molar | (8 hours reaction time) | | (20 hours reaction time) | |
| Entry | (° C.) | (gm) | ratio | RKunsat28 | RKunsat33 | RKunsat28 | RKunsat33 |
| 1 | 66 | 1.21 | 8 | 64.2 | 4.1 | | |
| 2 | 66 | 1.21 | 8 | 55.5 | | 91.0 | |
| 3 | 80 | 1.22 | 8 | 39.4 | | 80.1 | |
| 4 | 80 | 0.62 | 8 | 63.5 | | | |
| 5 | 66 | 0.63 | 8 | 27.1 | | 73.5 | |
| 6 | 66 | 0.60 | 8 | 62.1 | | | |
| 7 | 66 | 0.61 | 4 | 18.6 | | | |
| 8 | 64 | 1.24 | 16 | 67.5 | | | |
| 9 | 67 | 1.24 | 8 | 71.3 | 2.8 | 85.8 | 10.3 |
| 10 | 67 | 1.20 | 8 | 74.7 | 3.1 | 84.0 | 5.8 |
| 11 | 64 | 1.24 | 4 | | | 39.5 | |
| 12 | 66 | 1.23 | 4 | | | 43.7 | |
| 13 | 63 | 0.61 | 4 | 19.2 | | 40.9 | |
| 14 | 66 | 0.63 | 16 | 63.6 | | 90.3 | |
| 15 | 66 | 0.63 | 4 | | | 51.7 | |

FF = furfural; Tri = 12-trocosanone. Methanol was used a solvent.

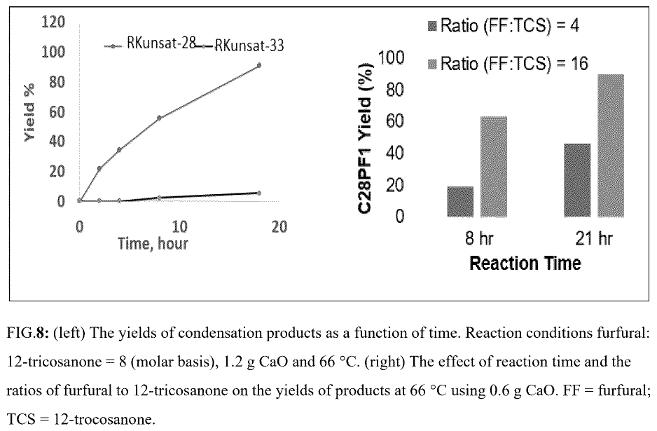

FIG. 8 shows conversion of 12-trocosanone and the yields of Rkunsat-28 and RKunsat-33 as a function of reaction time. Additional experiments using two different molar ratios of the reactants (furfural: 12-trocosanone=4 and 16), but with a fixed amount of CaO (0.6 g) indicate that the yield of RKunsat-28 increased as a function of time in both cases. The yield is significantly lower at lower ratio of the reactants (FIG. 8). When the furfural to 12-tricosanone molar ratio was 4, the yield of RKunsat-28 was 46% at 21 hour. The effect is more pronounced when the reactant molar ratio was increased from 4 to 16. The yield increased to 90% during the same time. The reaction rate is faster (the yield is higher at a given reaction time) with higher molar ratio of 16 due to increased concentration of furfural in the reaction mixture. It was also observed during the course of the reactions that no RKunsat-33 product was formed using 0.6 g catalyst; however, a small amount of (5%) of RKunsat-33 was formed during the same reaction scale when 1.2 g CaO was used.

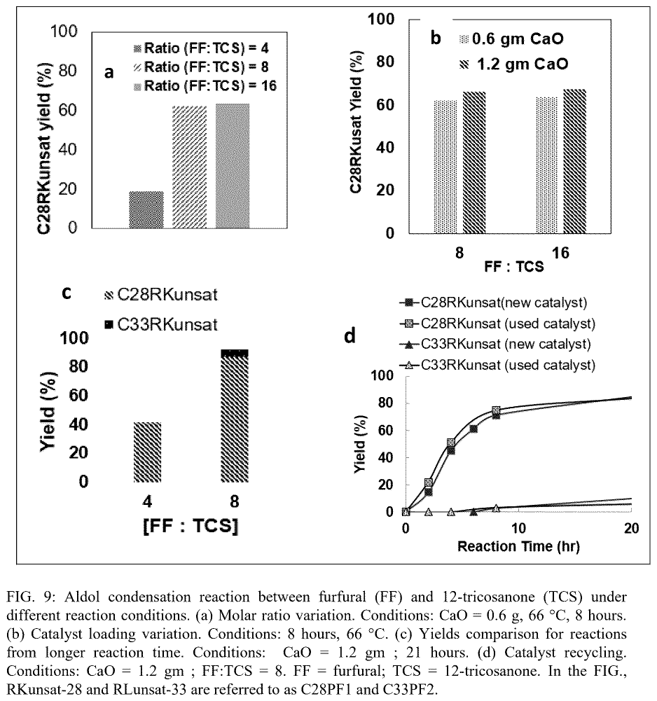

The yield of RKunat-28 at furfural to 12-tricosanone molar ratio of 8 is similar to that of molar ratio of 16 (FIG. 9a), which is likely because of reaching an equilibrium of the reaction at the molar ratio of 8. The yield increased from 62% at molar ratio of 8 to 64% at molar ratio of 16 for 8 hours using 0.6 g CaO catalyst at 66° C. The results suggested that the gain in the reaction rate by increasing furfural concentration beyond the furfural and 12-tricosanone molar ratio of 8 would be rather small. FIG. 9b shows that the benefit of having higher molar ratio of 16 on the RKunsat-16 yield is rather nominal. This is true at both low and high levels of CaO catalyst.

It is also clear from FIG. 9b that the yield of RKunsat-28 does not change significantly by doubling the catalyst amount. The yield of RKunsat-28 increased from 62% to 67% for low (0.6 μm) to high (1.2 μm) levels of catalyst. In the presence of higher amounts of CaO (1.2 g), a small amount of RKunat-33 was formed at furfural to 12-tricosanone molar ratio of 8 for 21 hours (FIG. 9C). The yield of RKunsat-33 was not observed at furfural to 12-tricosanone molar ratio of 4 (FIG. 9c), even after 21 hours of reaction. Mechanistically, base catalyzed aldol condensation proceeds via enolate formation (as an intermediate.

CaO catalyst was recovered by filtration from a reaction conducted between furfural and 12-tricosanone at furfural to 12-tricosanone molar ratio of 8 at 66° C. for 21 hours using 1.2 g catalyst. The recovered catalyst was filtered, washed with methanol and dried in a furnace for reusing in the next cycle. Because there was a loss of some catalyst (0.5 g) during filtration, washing, and drying, we added 0.5 g fresh CaO to the recovered CaO for carrying out the reaction in the next cycle between furfural and 12-tricosanone under identical conditions. FIG. 9d shows the yields of C₂₈PF₁ and C₃₃PF₂ products as a function of reaction time. The profiles of both products are very similar for new catalyst and the recovered catalysts containing 0.5 g new catalyst. It suggests that CaO deactivation may not be a major issue for the aldol condensation reaction.

Scalability demonstration (continue Table 3, entry 16). A larger scale reaction was conducted using CaO as the catalyst. 100 g (0.297 mol) 12-tricosanone, 228 g (2.38 mol) furfural, 75 g CaO and 1 L methanol were used. Tricosanone to furfural molar ratio was 8. The reaction was done in the 10 L jacketed glass reactor under comparable conditions discussed above. The bath temperature was set at 80° C. and the actual bulk reaction temperature was 68° C. First, 1 L methanol and 12-tricosanone were added to the reactor. The mixture was stirred at 200 rpm stirring rate. Furfural was added when the temperature reached about 55° C. Tricosanone dissolved completely upon addition of furfural. Then 75 g CaO was added to 100 mL methanol to make a slurry in a beaker and the slurry was added into the reactor. The samples were collected at 0 hour, 6 hour, 21 hour and 26 hour. The conversion of tricosanone was quantitative. The yields of RKunst-28 and RKunsat-33 increased as a function of time. After 21 hours reaction, total yield was 94% (85% RKunsat-28 and 8% RKunsat-33). After 26 hours, selectivity to RKunst-28 and RKunsat-33 were 88% and 11%, respectively. The results show the reaction profile under the scaled-up conditions was similar to that of small-scale reaction and that the product quality is comparable to the small-scale reaction. Thus, the process is scalable.

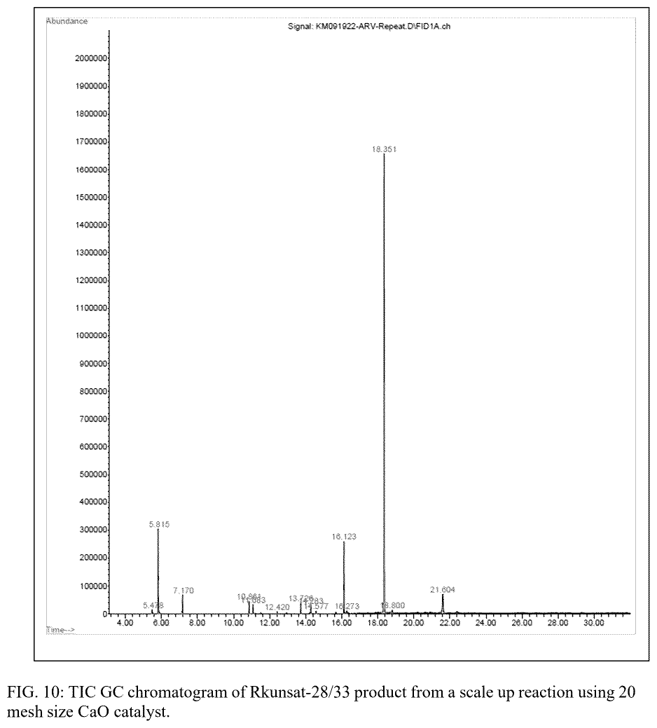

(Continue Table 3, entry 17) Another reaction was performed using CaO catalyst of particle size 20 mesh (850 micron). 150 g tricosanone (0.44 mol), 228 g furfural (2.374 mol), 1.5 L methanol and 75 g CaO were used in this reaction. The particle size of CaO catalyst was 20 mesh. The reaction methodology in terms of adding reactants and solvent was the same as described above. This reaction took a significantly longer time (70 hours) to finish the reaction, which could be due to the degradation of the catalyst in the presence of moisture or for larger size of the catalyst particles. The RKunsat-28/33 product was rotary evaporated to separate any remaining furfural, solvent and furfural degraded furan methanol. The final RKunsat-28/33 product contained 71% RKunsat-28, 6% RKunsat-33, 9% furan methanol and 6% unconverted 12-tricosanone (FIG. 10).

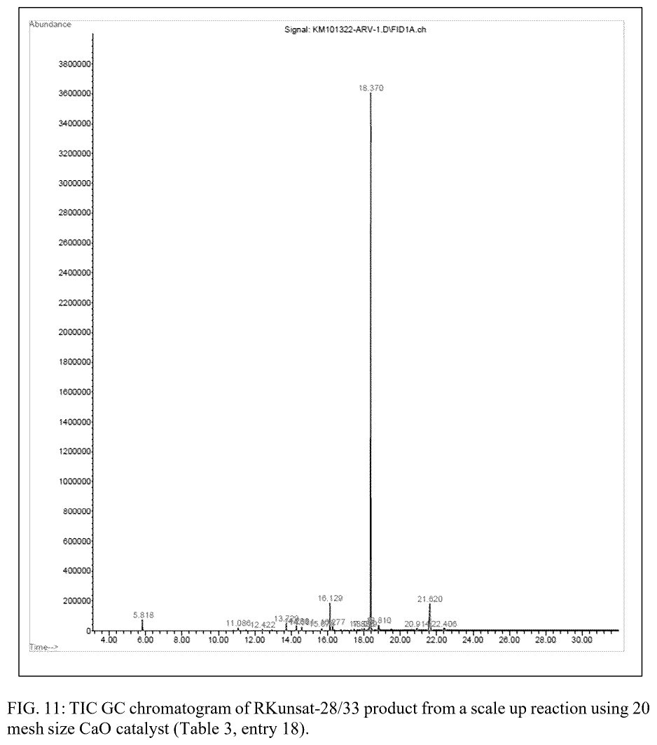

(Continue Table 3, entry 18) Another aldol condensation reaction under the same condition as entry 17 and using 20 mesh size CaO catalyst was conducted for 88 hours. After rotary evaporation of solvent, unconverted furfural and furan methanol, the final RKUnsat-28/33 product contained 83% RKunsat-28 and 8.5% RKunsat-33 as the major product along with 1.4% furan methanol and other small peaks (FIG. 11).

### Example 4. Preparation of RKunsat-14

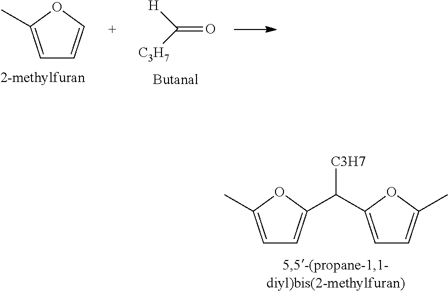

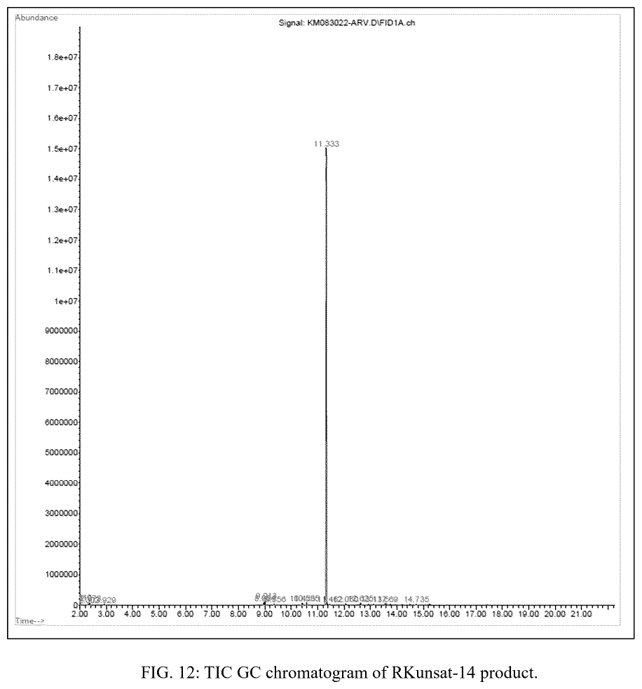

RKunsat-14 was prepared by catalytic condensation between commercially procured biomass derived 2MF and butaldehyde using P—SiO2 as the catalyst in the temperature range of 60° C. to 77° C. The molar ratio of 2MF and butaldehyde was in the range of 3-4. The experimental and product purification methodologies were similar to those of RKunsat-17 and RKunsat-22 products. The reaction temperature was slowly increased to avoid 2MF polymerization in the beginning of the reaction. After completion of the reaction and colling down the reaction mass, the catalyst was separated by filtration and the product was distilled off to separate any unconverted 2MF and butaldehyde. Table 4 summarizes representative results. After rotary evaporation, the purity of RKunsat-14 was >98%. FIG. 12 shows GC chromatogram of the product indicating RKunsat-14 is the only product after the reaction.

TABLE 4
Production of RKunsat-14 on different scales under various reaction conditions.

| | Reaction | | Yield of | Conv of |
|---|---|---|---|---|
| Entry # | scale | Catalyst & reaction conditions | RKunsat-14 | butyldehyde |
| 1 | 540 g | P-SiO2; 60° C. for 0.5 h, 70° C. for 0.5 h, | 81% | 84% |
| | | 75° C. for 3.5 hours | | |
| 2 | 120 g | P-SiO2 (10 g); 60° C. for 2 h, 70° C. for | 98% | 90% |
| | | 2 h, 75° C. for 6 h | | |
| 3 | 320 g | P-SiO2 (40 g); 60° C. for 1 h, 75° C. for | 78% | 81% |
| | | 3 h | | |
| 4 | 120 g | P-SiO2 (11 g); 50° C. for 0.5 h, 60° C. | 97% | 97.5% |
| | | for 0.5 h; 65-80° C. for 12 h | | |

Example 5. Hydrodeoxygenation of RKunsat-22 of RKBA-22 in the flow reactor. RKunsat-22 was first hydrogenated in the flow reactor to produce furan ring hydrogenated intermediate and then hydrogenated intermediate was separately passed through the same catalyst bed to deoxygenate the hydrogenated feed to RKBA-22.

### Furan Ring Hydrogenation of RKunsat-22 to RKsatOH-22 (6-undecylundecane-2,10-diol) and RKsat-22 (2-methyl-5-[1-(5-methyloxolan-2-yl)dodecyl]oxolane)

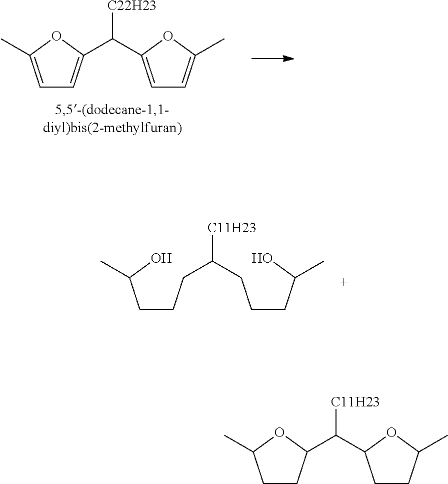

Furan ring hydrogenation of RKunsat-22 feed was performed in the flow reactor of ⅜″ OD. An oil bath was used for heating the catalyst bed and transferring excess heat from the catalyst bed. Flow rate was varied to determined magnitude of heat of exotherm as a function of flow rate of feed. The temperature of the catalyst bed and the bath temperature were similar at 0.25 ml/min flow rate of the feeds stream. The reaction parameters were varied as shown in Table 5.

TABLE 5
Reaction conditions and process parameters for hydrogenation of RKunsat-22 in the
tubular reactor.

| Parameter | Value | Units | Comments |
|---|---|---|---|
| Reactor OD | 0.375 | in | 316 L SS Tubing |
| Reactor ID | 0.305 | in | |
| Active Packing Length | 12 & 18 | in | 250-700 μ size catalyst particles were mixed |
| Weight of catalyst (Wcat) | 1.59 & 2.44 | g | with similar size sand |
| Bed Dilution | 8.3, 16 | % | |
| (Wt% of catalyst) | | | |
| Oil bath temperature | 150, 160, 170, | ° C. | Julabo oil heater with heating cartridges |
| | 180 | | |
| Reactor pressure | 600, 400 & 200 | psig | This pressure is provided by the reactor |
| | | | Equilibar BPR |
| Wt % of RKunsat-22 in | 50, 75 & 100 | % | Appropriate amount of decane was used to |
| Feed | | | make the diluted feed. (100% refers to pure |
| | | | feed) |
| Liquid flow rate (Qliq) | 0.18, 0.25, 0.36, | ml/min | Liquid flowrate through the Eldex pump |
| | 0.5, 1 & 2 | | |
| Mass flow of H2 | 3 | g/h | Setpoint on the MFC |
| LWHSV | 5 to 45 | $\frac{{g}_{reac}}{h \cdot {g}_{cat}}$ | Estimated from mass flow of RKunsat-22 and catalyst weight |
| GWHSV | 1.89 & 1.23 | $\frac{{g}_{{H}_{2}}}{h \cdot {g}_{c a t}}$ | Estimated from mass flow of H₂ and catalyst weight |
| Liquid superficial velocity | 0.01 to 0.07 | cm/s | $Estimated by using the correlation {U}_{s}^{L i q} =$ |
| $( {U}_{s}^{L i q} )$ | | | $\frac{{Q}_{liq}}{C . S . A . of the reactor}$ |
| Gas superficial velocity @ | ~0.5 | cm/s | $Estimated by using the correlation {U}_{s}^{Gas} =$ |
| $600 psi and 25 ° C . ( {U}_{s}^{Gas} )$ | | | $\frac{{Q}_{gas}}{C . S . A of the reactor}$ |
| Liquid mean residence time | 2.25 to 18 | min | Estimated by using the correlation τ = |
| | | | $\frac{{εV}_{bed}}{{Q}_{liq}} at various liquid flowrates while$ |
| | | | assuming a bed voidage (ε) of 0.3 |

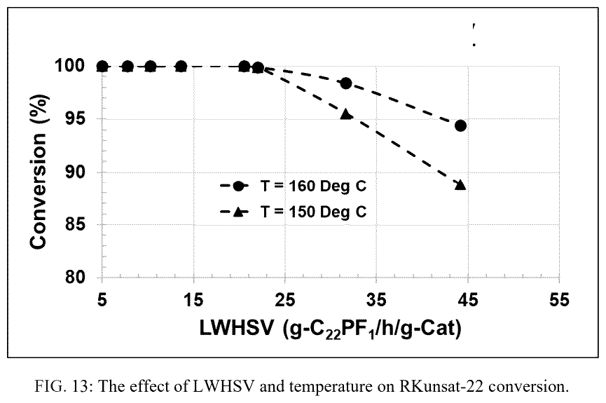

GC and GCMS analyses indicated complete conversion of RKunsat-22 and 95% yield of fully furan-ring hydrogenated intermediate (RKsat-22) and RKsatOH-22 at 150° C. and 160° C. A small amount of single furan ring hydrogenated intermediates were also formed. FIG. 13 results show that complete conversion of C₂₂PF₁ was achieved for low LWHSV values

$$( 5 - 25 \frac{{g}_{reac}}{h . {g}_{cat}} )$$

at both 150° C. and 160° C. As the LWHSV was increased beyond

$$25 \frac{{g}_{reac}}{h . {g}_{cat}} ,$$

some unreacted feed was identified in the product. Additional experiments were performed in the tubular reactor with 18″ of active packing of the catalyst containing 16 wt % catalyst and 84% inert material at varying reaction parameters. Table 6 summarized the representative results.

TABLE 6
The yields of RKunsat-22 hydrogenated product at different
reaction parameters in the tubular reactor.

| | | | | | | | Total yield |
|---|---|---|---|---|---|---|---|
| | Cat | | | | | | of RKsat22 |
| | bed | | H2 | Cat | | | and |
| | temper- | | flow | bed | | Conv of | RKsatOH- |
| Entry | ature | Pressure | rate | length | | RKunsat- | 22 |
| No. | (C) | (psi) | (g/h) | (inch) | LWHSV | 22 (%) | (%) |
| 1 | 150 | 200 | 3 | 12 | 6.9 | 100 | 97 |
| 2 | 150 | 400 | 3 | 12 | 6.9 | 100 | 97 |
| 3 | 160 | 200 | 3 | 12 | 6.9 | 100 | 96 |
| 4 | 160 | 400 | 3 | 12 | 6.9 | 100 | 96 |
| 5 | 160 | 600 | 3 | 18 | 2.69 | 100 | 90 |
| 6 | 170 | 80 | 3 | 18 | 1.89 | 100 | 84 |
| 7 | 170 | 80 | | 18 | 3.78 | 100 | 86 |
| 8 | 170 | 600 | 3 | 18 | 2.63 | 100 | 90 |
| 9 | 175 | 600 | 3 | 18 | 2.63 | 100 | 92 |
| 10 | 180 | 80 | 3 | 18 | 3.78 | 100 | 84 |

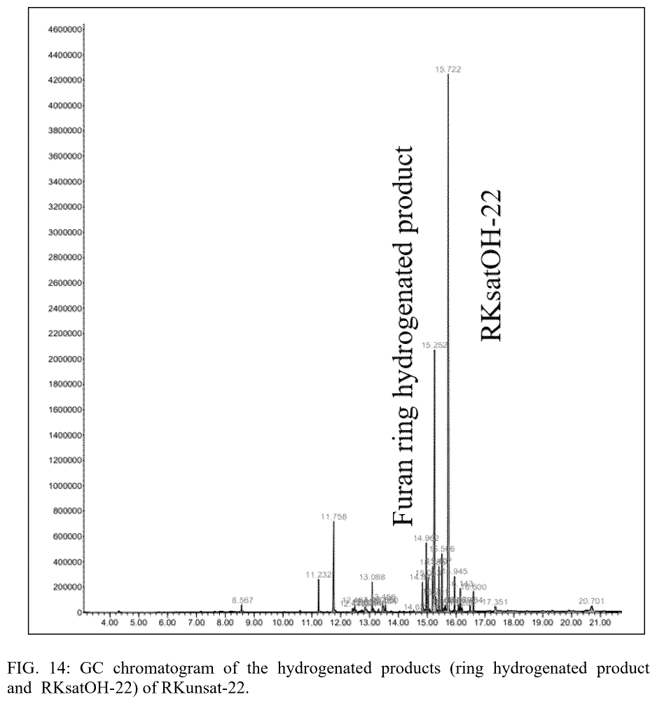

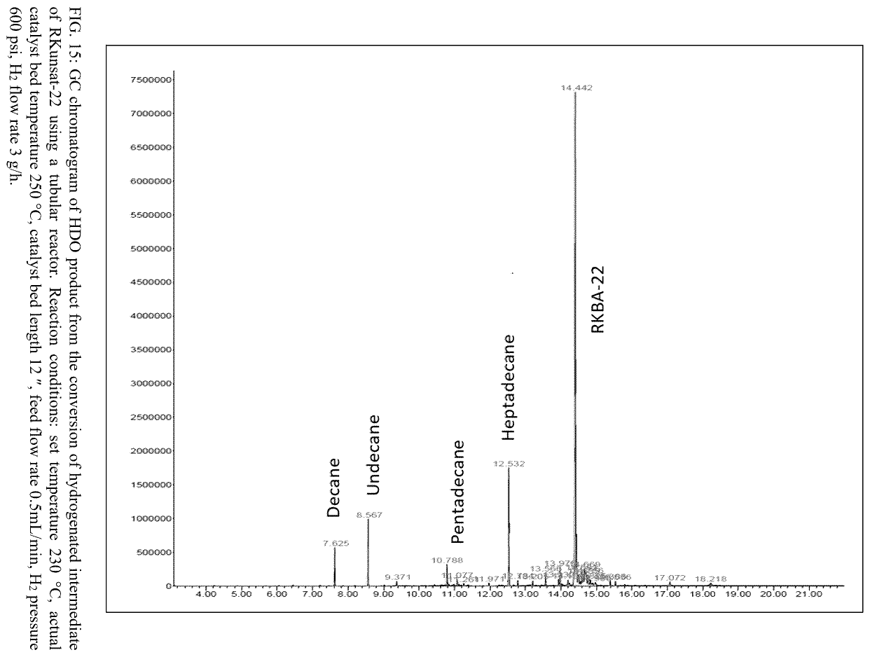

Experiments were conducted in batch reactor to hydrogenated RKunsat-22. In case of batch reactor using Ni62/15P as the catalyst at 5 bar of H₂ pressure and 160° C., the majority of the hydrogenated product was RKsatOH-22. Total reaction time was 4 h. After completion of the reaction, the reactor was cooled down, the reactor was depressurized, and the product was vacuum filtered from the reactor. The dominant peaks (retention time between 15.2 min and 15.7 min) in the GC chromatogram (FIG. 14) correspond to RKsat-22 and ring opened RKsatOH-22 of RKunsat-22. Up to 82% yield of total hydrogenated intermediate species was obtained of which RKsatOH-22 is >60%. The results obtained from the Parr reactor clearly suggest that the tubular reactor system provided better yield and selectivity towards the hydrogenated intermediates owing to enhanced mass and heat transfer phenomena.

Deoxygenation of furan ring hydrogenation intermediates of RKunsat-22 to RKBA-22.

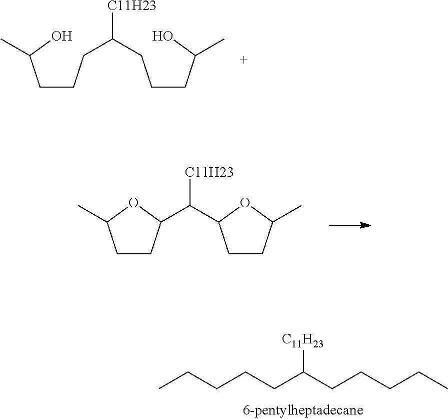

The fully hydrogenated product of RKunsat-22 was reacted in the tubular reactor to perform the deoxygenation chemistry to produce RKBA-22. The bed length of the catalyst bed containing 20% PRICAT Ni 60/15T crashed catalyst pellet of 250-700 micron size and 80% inert was 12″. A tubular furnace with 18″ of heated zone was used for heating the catalyst bed. The set temperature of the furnace was 230° C. The hydrogenated intermediate product was fed to the catalyst bed at a flow rate of 0.5 mL/min. H₂ gas flow rate was 3 g/h at a set pressure of 600 psi. At set furnace temperature of 230° C., the temperature of the catalyst bed was 250° C. indicating an exotherm of about 20° C. The product was collected and analyzed by GCMS. The results (FIG. 15) show full deoxygenation of the hydrogenated intermediate feed. The yield of RKBA-22 was 66%. The balance was C—C cleavage product. Among the cleavage product, heptadecane was the major fraction (11%). Undecane (6%), pentadecane (2%), nonadecane (1.2%) and other small amounts of C16-C21 alkanes were detected. About 3% unconverted hydrogenated feed was also detected. The reaction was not inhibited by water by-product that is formed during the reaction.

Example 6. Hydrodeoxygenation of RKunsat-17 of RKBA-17 in the flow reactor. RKunsat-17 was first hydrogenated in a Parr reactor to produce furan ring hydrogenated intermediate and then hydrogenated intermediate was fed to the flow reactor to deoxygenate the hydrogenated feed to RKBA-17.

### Furan Ring Hydrogenation and Ring-Opening of RKunsat-17 to RKsatOH-17 (6-hexylundecane-2,10-diol) and RKsat-17 (2-methyl-5-[1-(5-methyloxolan-2-yl)heptyl]oxolane)

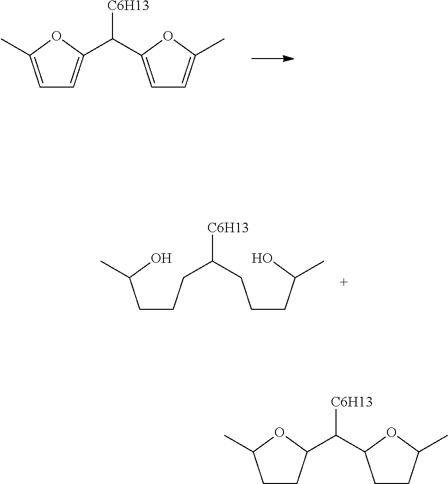

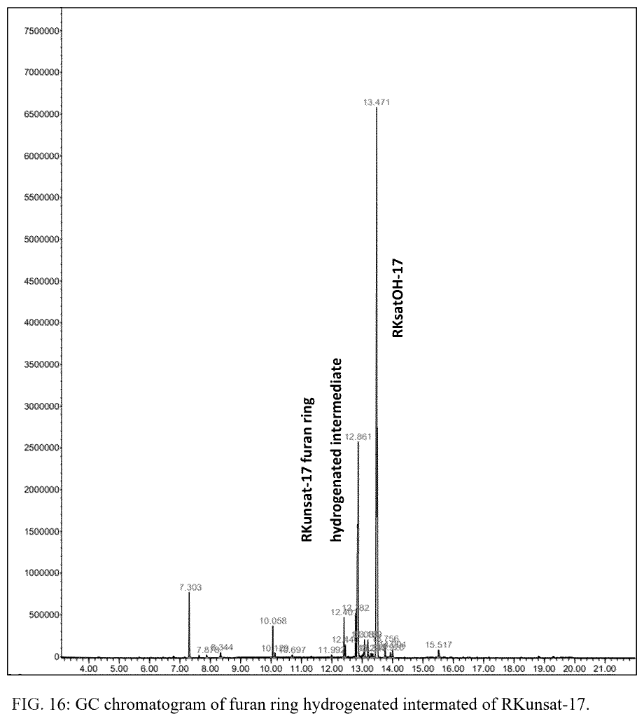

Furan ring hydrogenation and ring opening of RKunsat-17 was performed in a 2 L Parr reactor at 160° C. and 45 bar of H₂ pressure in the presence of a commercial Ni-based catalyst, Ni62/15P. An internal cooling coil was used in the Parr reactor to control the reaction exotherm during the hydrogenation step. The FID chromatogram of the hydrogenated intermediate is shown in FIG. 16. The peaks with retention time of 12.861 min and 13.471 min represent the peaks for RKsat-17 and RKsatOH-17. The majority of the product (>70%) was ring opened RKsatOH-17. The effect of pressure and reaction time on the hydrogenation chemistry was studied in the Parr reactor system and the relevant key results along with the process conditions are shown in Table 7. It was observed that the yields of the intermediates (RKsatOH-17 and RKsat-17) remained the same at varying conditions indicating a pseudo-first order kinetic behavior. A small amount of other intermediates were formed. This product was then used as a feed in the tubular reactor to conduct hydrodeoxygenation to produce RKBA-17.

Deoxygenation of furan ring hydrogenation intermediates of RKunsat-17 to RKBA-17.

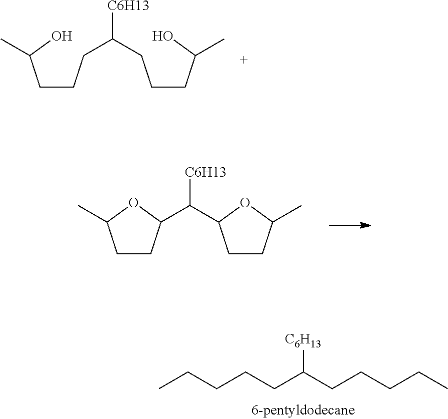

The reaction was performed in the tubular reactor of 2 ft long and ⅜″ OD SS equipped with a tubular furnace having a heated zone of 18′. The reactor was packed with the Ni62/15P catalyst of 10 Wt % of 100-250μ size catalyst particles mixed with similar size sand. A thermocouple was inserted to the center of the catalyst bed to monitor the reaction exotherm. The flowrate of the hydrogenated feed was slowly increased at a constant furnace temperature to control the exotherm and ensure that the catalyst bed temperature is stable. The reaction conditions and process parameters are shown in Table 7. The catalyst bed was activated using H₂, wetted and preheated prior to fed the hydrogenated intermediate of RKunsat-17.

TABLE 7
The yields of RKunsat-17 hydrogenated product at different reaction parameters.

| | | | | | Total yield of |
|---|---|---|---|---|---|
| | | | Conversion of | Reaction | RKsatOH-17 |
| | Temperature | | RKunsat-17 | time | and RKsat-17 |
| Entry | (C.) | Pressure (psi) | (%) | (h) | (%) |
| 1 | 160 | Step 1: 5 bar for 1 hr | 100 | 5 | 84.6 |
| | | Step 2: 35 bar for 4 hr | | | |
| 2 | | Step 1: 5 bar for 1 hr | 100 | 4 | 84.1 |
| | | Step 2: 35 bar for 3 hr | | | |
| 3 | | Step 1: 10 bar for 1 hr | 100 | 6 | 81.6 |
| | | Step 2: 35 bar for 5 hr | | | |

TABLE 8
Reaction conditions and process parameters for all the tubular reactor experiments.

| Parameter | Value | Units | Comments |
|---|---|---|---|
| Reactor OD | 0.375 | in | 316 L SS Tubing |
| Reactor ID | 0.305 | in | |
| Active Packing Length | 6 | in | 100-250 μ size catalyst particles |
| Weight of catalyst (W_{cat}) | 2.03 | g | were mixed with similar size |
| Bed Dilution | 10 | % | sand |
| (Wt % of catalyst) | | | |
| Reactor pressure | 600 | psig | This pressure is provided by the |
| | | | reactor Equilibar BPR |
| Furnace temperature | 230, 240, 250 & | ° C. | The setpoint was adjusted based on |
| | 255 | | the observed bed temperature |
| Liquid flow rate (Q_{liq}) | 0.1, 0.2 & 0.4 | ml/min | Liquid flowrate through the Eldex |
| | | | pump was gradually increased from |
| | | | 0.1 to 0.4 ml/min to control the |
| | | | exotherm |
| Mass flow of H₂ | 3 | g/h | Setpoint on the MFC |
| LWHSV | 10.6 | $\frac{{g}_{r e a c}}{h \cdot {g}_{c a t}}$ | Estimated from mass flow of C17sat and catalyst weight |
| GWHSV | 1.47 | $\frac{{g}_{{H}_{2}}}{h \cdot {g}_{c a t}}$ | Estimated from mass flow of H₂ and catalyst weight |
| Liquid superficial velocity | 0.01 | cm/s | Estimated by using the correlation |
| $( {U}_{s}^{L i q} )$ | | | ${U}_{s}^{L i q} = \frac{{Q}_{liq}}{C . S . A of the reactor}$ |
| Gas superficial velocity @ | ~0.5 | cm/s | Estimated by using the correlation |
| $600 psi and 25 ° C . ( {U}_{s}^{Gas} )$ | | | ${U}_{s}^{G a s} = \frac{{Q}_{gas}}{C . S . A of the reactor}$ |
| Liquid mean residence time | ~5 | min | Estimated by using the correlation |
| | | | $τ = \frac{{εV}_{bed}}{{Q}_{liq}} at various liquid$ |
| | | | flowrates while assuming a bed |
| | | | voidage (ε) of 0.3 |

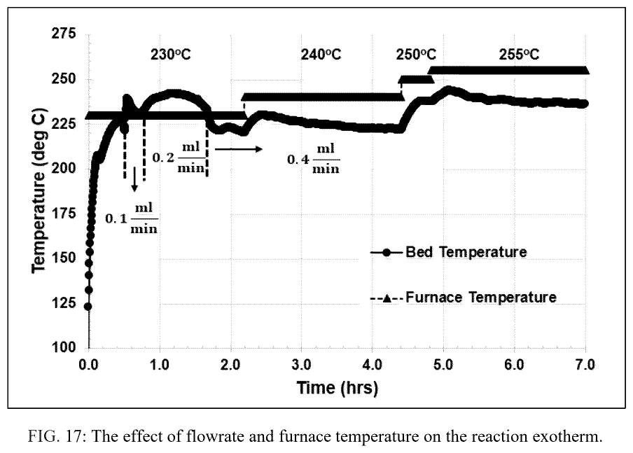

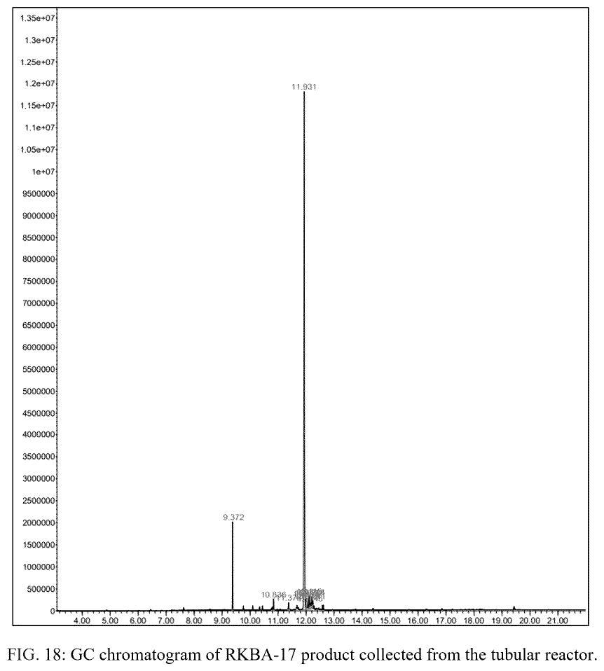

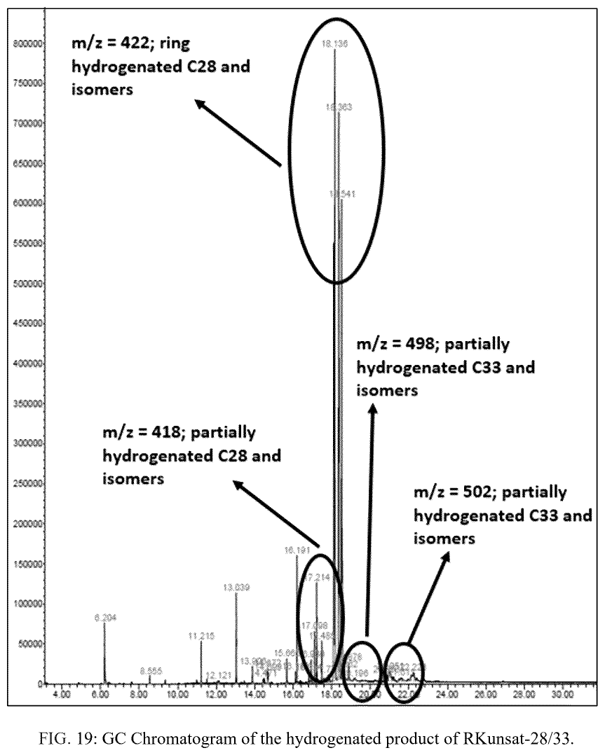

The furnace was initially set at 230° C. and the feed flowrate was set at 2 ml/min during the preheating and catalyst wetting step. Once the reactor was heated and the catalyst bed was wetted with the liquid, H₂ was introduced into the reactor at 3 g/h and the liquid flowrate was reduced to 0.1 ml/min. The bed temperature was then closely monitored for to observe the behavior of the reaction exotherm. Once the temperature of the catalyst bed stabilized, the flowrate was then increased to 0.2 ml/min and later to 0.4 ml/min while monitoring the bed temperature. The bed temperature profile and the furnace setpoints as a function of time is shown in FIG. 17. Once the reactor was operating at the desired flowrate of 0.4 ml/min, it was observed was that the bed temperature started to drop below the furnace temperature of 230° C. To counter this heat loss, the furnace temperature was then increased sequentially to maintain the bed temperature at ~240° C. The average bed temperatures during the furnace setpoint ramp up procedure while the reactor was operating at a constant flow of 0.4 ml/min are shown in Table 9. The final HDO product was collected after the recorded bed temperature was >230° C. Water co-product was phase separated in the collection vessel. The reaction was not inhibited by the water co-product. The FID chromatogram and the respective area percentages of the detected peaks in the final product are shown in FIG. 18. The yields of RKBA-17 and a C—C cleavage dodecane were 84% and 8.3%, respectively. Dodecane was distilled off to obtain RKBA-17 with high purity.

TABLE 9
Average bed temperature at various furnace
setpoints with the reactor operating at a
constant liquid flow of 0.4 ml/min.

| Furnace | | Average Bed |
|---|---|---|
| Temperature | Flowrate | Temperature |
| (° C.) | (ml/min) | (° C.) |
| 230 | 0.4 | 226.3 |
| 240 | | 224.8 |
| 250 | | 233.4 |
| 255 | | 238.3 |

The process was then scaled up in the tubular reactor of 1″ OD using the best conditions. The 1″ tube was packed with as received PRICAT Ni 60/15T catalyst palette with a 10% and 50% dilution using an inert material. The length of the catalyst packing was 10″. The flowrate of the feed stream was varied in the range of 0.5-2 mL/min at different furnace temperature set points. It was found that the reaction at 0.75 mL/min flowrate, in a 10% diluted catalyst bed, gave good product with selectivity to the desired branched alkane of 70% with balance being C—C cleavage alkanes. Also, it was found that the reaction at 0.8 ml/min of flowrate, in a 50% diluted catalyst bed, gave product with similar selectivity as in the 10% diluted catalyst bed. However, in all the experiments, the bed temperature was cooling off slowly at the lower end of the catalyst bed as the reaction was progressing possibly due to water formation. Thus, the furnace set temperature needed to increase slowly to offset the temperature loss. For example, a reaction at 0.8 mL/min was started at the set furnace temperature of 240 C and the temperature was slowly increased to 270 C at 2.6 hour of the reaction to maintain a constant temperature of the catalyst bed of 240 C. The time-of-stream study of the catalyst indicated that the catalyst was active up to the operational time of 200 hours. The experimental conditions used for these experiments are given in Table 10. Also, the effect of process parameters on the conversion and yield across all the tubular reactor experiments are shown in Table 11.

TABLE 10
Reaction conditions and process parameters for all the tubular reactor experiments.

| Parameter | Value | Units | Comments |
|---|---|---|---|
| Reactor OD | 1.00 | in | 316 L SS Tubing |
| Reactor ID | 0.834 | in | |
| Active Packing Length | 10 | in | PRICAT Ni 62/15T 5 mm × 4 mm |
| Weight of catalyst (W_{cat}) | 26.3 & 39.9 | g | cylindrical pellets mixed with |
| Bed Dilution | 10 & 50 | % | similar size alumina pellets |
| (Wt % of catalyst) | | | |
| Reactor pressure | 600 | psig | This pressure is provided by the |
| | | | reactor Equilibar BPR |
| Furnace temperature | 200-270 | ° C. | The setpoint was adjusted based on |
| | | | the observed bed temperature |
| Liquid flow rate (Q_{liq}) | 0.5-1.75 | ml/min | Liquid flowrate through the Eldex |
| | | | pump was gradually increased to |
| | | | control the exotherm |
| Mass flow of H₂ | 6-12 | g/h | Setpoint on the MFC |
| LWHSV | 0.7-3.6 | $\frac{{g}_{reac}}{h \cdot {g}_{cat}}$ | Estimated from mass flow of RK17sat and catalyst weight |
| GWHSV | 0.2-0.5 | $\frac{{g}_{{H}_{2}}}{h \cdot {g}_{c a t}}$ | Estimated from mass flow of H₂ and catalyst weight |
| Liquid superficial velocity | 0.01-0.004 | cm/s | Estimated by using the correlation |
| $( {U}_{s}^{L i q} )$ | | | ${U}_{s}^{L i q} = \frac{{Q}_{liq}}{C . S . A of the reactor}$ |
| Liquid mean residence time | 15-50 | min | Estimated by using the correlation |
| | | | $τ = \frac{ε {V}_{b e d}}{{Q}_{liq}} at various liquid$ |
| | | | flowrates while assuming a bed |
| | | | voidage (ε) of 0.3 |

TABLE 11
The yields of BA17 product at different reaction parameters in a tubular reactor
system.

| | | | Amount | | | | | Conversion | |
|---|---|---|---|---|---|---|---|---|---|
| | | Catalyst | of | | | | | of hydrogenated | Yield |
| | | bed | catalyst | Furnace | H₂ | Liquid | | intermediates | of |
| | Reactor | length | in bed | temperature | flowrate | flowrate | | RKSatOH- | BA17 |
| Entry | OD (in) | (in) | (wt%) | (° C.) | (g/h) | (ml/min) | LWHSV | 17) (%) | (%) |
| 1 | 0.375 | 6 | 10 | 230-255 | 3 | 0.4 | 10.6 | 100 | 84 |
| 2 | 1 | 10 | 10 | 250-290 | 12 | 1.75 | 3.6 | 38.2 | 20.6 |
| 3 | 1 | 10 | 10 | 220-250 | 12 | 0.75 | 1 | 86.5 | 51.3 |
| 4 | 1 | 10 | 50 | 240 | 12 | 1 | 1.4 | 95.5 | 45.5 |
| 5 | 1 | 10 | 50 | 240 | 6 | 0.5 | 0.7 | 83.4 | 26.4 |
| 6 | 1 | 10 | 50 | 240-270 | 6 | 0.8 | 1.1 | 84.8 | 54.3 |

Example 7. Hydrodeoxygenation of RKunsat-28/33 of RKBA-28/33 in the flow reactor using cyclohexane as a solvent. RKunsat-28/33 was first hydrogenated in the Parr reactor to produce furan ring hydrogenated intermediate and then hydrogenated intermediate was fed to the flow reactor to deoxygenate the hydrogenated feed to RKBA-28/33.

Furan ring hydrogenation of RKunsat-28/33.

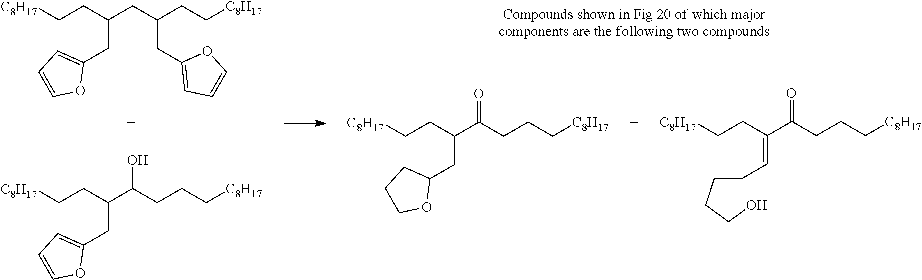

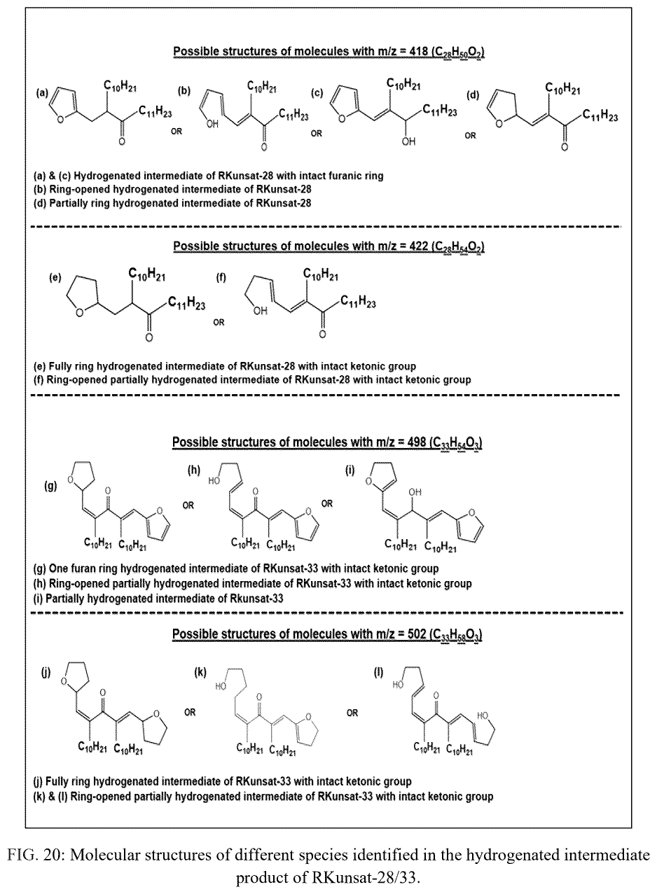

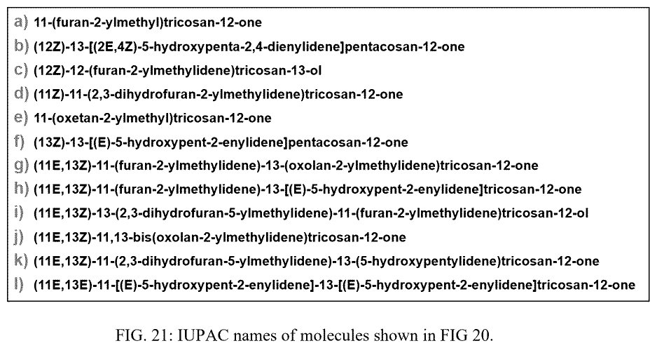

Furan ring hydrogenation of RKunsat-28/33 was done in the Parr reactor using cyclohexane as a solvent and Ni62/15P as the catalyst. H₂ pressure was 660 psi (~45 bar) under ambient conditions. The pressure was balanced whenever the reactor pressure dropped below 660 psi. Total reaction time was 4 h. After completion of the reaction, the reactor was cooled down, the reactor was depressurized, and the product was vacuum filtered from the reactor. Solvent was separated by rotary evaporation. The dominant peaks (RT >18 min) in the GC chromatogram (FIG. 19) correspond to the ring hydrogenated intermediates from RKunsat28 and possible olefinic intermediates with C—C cleavage of RKunsat-33. Up to 92% yield of total hydrogenated intermediate species was obtained of which major components are C28 species containing ring hydrogenated furan and ring-opened hydroxyl functional group. The represented species identified in the hydrogenated product and the corresponding IUPAC names are shown in FIG. 20 and FIG. 21, respectively. The yield of two major hydrogenated species was up to 72%.

Deoxygenation of furan ring hydrogenation intermediates of RKunsat-28/33 to RKBA-28/33.

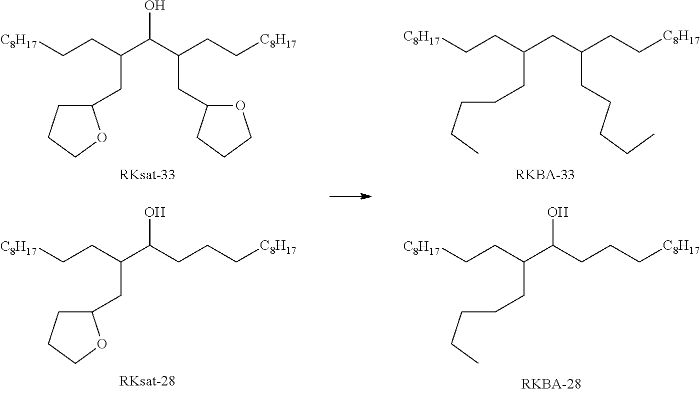

The hydrogenated intermediate product of RKunsat-28/33 was diluted in cyclohexane (~50 wt %), to enhance H₂ dissolution, and fed to the tubular reactor of 2 ft long 3/8-in OD SS tube heated by a tubular furnace with a heated zone of 18-in. The reactor was packed with 10-inches of diluted catalyst packing containing 20 Wt % of 700-250μ size catalyst particles mixed with similar size sand. A thermocouple was inserted to the center of the catalyst bed to monitor the reaction exotherm. The reaction conditions and process parameters are shown in Table 12.

TABLE 12
Reaction conditions and process parameters for deoxygenation of RKunsat-28/33
hydrogenated intermediate feed.

| Parameter | Value | Units | Comments |
|---|---|---|---|
| Reactor OD | 0.375 | in | 316 L SS Tubing |
| Reactor ID | 0.305 | in | |
| Active Packing Length | 10 | in | 250-700 μ size catalyst particles were mixed |
| Weight of catalyst (W_{cat}) | 4.9 | g | with similar size sand |
| Bed Dilution | 19.8 | % | |
| (Wt% of catalyst) | | | |
| Reactor pressure | 600 | psig | This pressure is provided by the reactor |
| | | | Equilibar BPR |
| Furnace temperature | 260 | ºC | The setpoint was adjusted based on the |
| | | | observed bed temperature |
| Liquid flow rate (Q_{liq}) | 0.8 | ml/min | Liquid flowrate through the Eldex pump was |
| | | | gradually increased to control the exotherm |
| Mass flow of H₂ | 4 | g/h | Setpoint on the MFC |
| LWHSV | 8.8 | $\frac{{g}_{reac}}{h \cdot {g}_{cat}}$ | Estimated from mass flow of RKunsat-28/33 and catalyst weight |
| GWHSV | 0.8 | $\frac{{g}_{{H}_{2}}}{h \cdot {g}_{cat}}$ | Estimated from mass flow of H₂ and catalyst weight |
| Liquid superficial velocity | 0.03 | cm/s | $Estimated by using the correlation {U}_{s}^{L i q} =$ |
| $( {U}_{s}^{L i q} )$ | | | $\frac{{Q}_{liq}}{C . S . A . of the reactor}$ |
| Liquid mean residence time | 4.5 | min | $Estimated by using the correlation {U}_{s}^{L i q} =$ |
| | | | at various liquid flowrates while assuming a |
| | | | bed voidage (ε) of 0.3 |

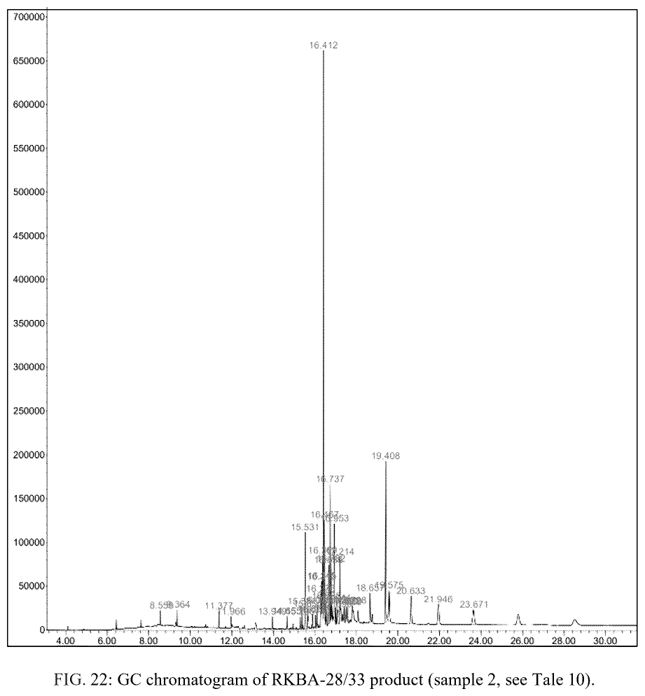

Prior to start the actual deoxygenation reaction, the catalyst bed was pre-reduced for 2 hrs at 160° C., 200 psi of reactor pressure and 12 g/h of H₂ flow. After catalyst activation, the furnace was set to 200° C. and the flowrate of the diluted hydrogenated feed was set at 2 ml/min during the preheating and catalyst wetting step. The preheating and catalyst wetting steps were performed in the absence of H₂. Once the reactor was heated and the catalyst bed was wetted with the liquid, H₂ was introduced into the reactor at 4 g/h and the feed flowrate was reduced to 0.4 ml/min. The bed temperature was then closely monitored to observe the behavior of the reaction exotherm. Once the temperature of the catalyst bed stabilized, the furnace temperature was increased to 240° C. while maintaining the flowrate of feed at 0.4 ml/min. The product collected at 240° C. set temperature (200° C. catalyst bed temperature) was colorless. The flow rate was then increased to 0.8 ml/min while carefully adjusting the furnace temperature so that the bed temperature was maintained at ~200° C. The product collected at both the flowrates was colorless. Table 13 summarizes key parameters and total yields of RKBA-28/33 products and other C—C cleaved compounds that were identified. FIG. 22 shows a representative GC profile of the product. It can be clearly observed, from the average catalyst bed temperature shown in Table 13, that no exotherm was detected under all the experimental conditions as the average bed temperature was always less than the furnace set point. The results indicated that C—C cleavage of C-28 and C-33 species occurred which resulted in the formation C-26 and C-30/31 branched hydrocarbons as the major species. The total yield of all identified hydrocarbon species of carbon numbers ≥C26 was up to 91%. The balance was hydrocarbons of carbon numbers lower than 26 of which C20 to C23 range hydrocarbon product was about 10%.

TABLE 13
The yields of RKBA-28/33 product at different reaction conditions in the tubular
reactor system.

| | | | | | | | Yield of total |
|---|---|---|---|---|---|---|---|
| | | | | | | | identified |
| | | | Average | | | | hydrocarbons |
| | | Furnace | Cat bed | | | | of carbon |
| | Flowrate | Temperature | temperature | Pressure | | Conv | numbers ≥26 |
| Entry | (ml/min) | (° C.) | (° C.) | (psi) | LWHSV | (%) | (%) |
| 1 | 0.4 | 220 | 201.8 | 600 | 2.7 | 100 | 89.6 |
| 2 | 0.4 | 230 | 209.4 | | 2.7 | 100 | 91.5 |
| 3 | 0.6 | 240 | 204.5 | | 4.05 | 100 | 90.8 |
| 4 | 0.8 | 250 | 199.3 | | 5.4 | 100 | 91.2 |

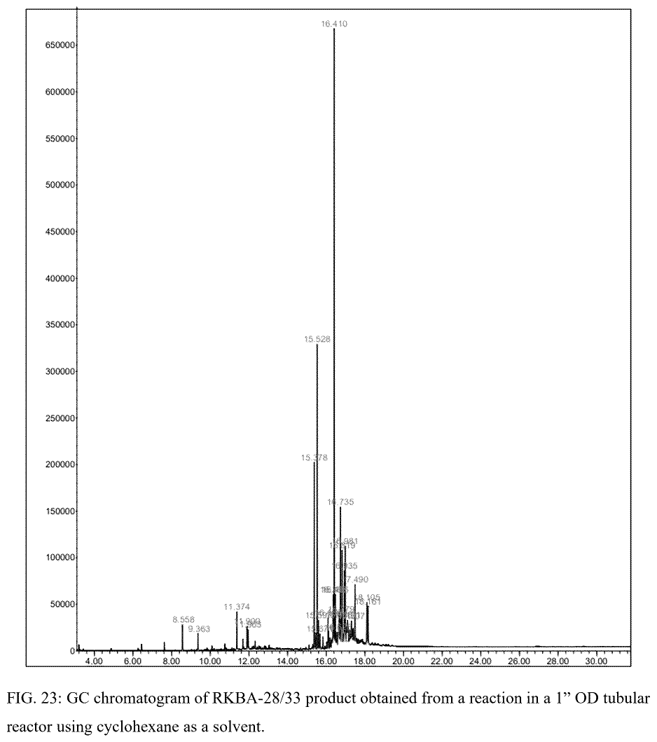

The process was then scaled up in the tubular reactor of 1″ OD using the best conditions. The reactor was packed with 14″ of diluted catalyst packing containing ~50 Wt % of PRICAT Ni 62/15T 5 mm×4 mm cylindrical pellets mixed with similar size alumina pellets. Three thermocouples were inserted into the top half (7-in) of the catalyst bed that measure the top, top middle and middle temperatures of the packing. The P&ID of the tubular reaction system and reactor packing methodology are shown in FIGS. 3 & 4. The process conditions are shown in Table 14. After catalyst activation, the furnace was set to 230° C., and the liquid flowrate was set at 2 ml/min during the preheating and catalyst wetting step. It must be noted that the preheating and catalyst wetting steps were performed in the absence of H₂. Once the reactor was heated and the catalyst bed was wetted with the liquid, H₂ was introduced into the reactor at 10 g/h and the liquid flowrate was maintained at 2 ml/min. The bed temperature was then closely monitored to observe the behavior of the reaction exotherm. Once the temperature of the catalyst bed stabilized, the furnace temperature was increased to 240° C. while maintaining the liquid flowrate at 2 ml/min. It can be clearly observed that no sustained exotherm was recorded and the average bed temperature was always less than the furnace set point. However, it must be noted that an initial hotspot was observed, at the TC2 location (See FIG. 4), during the reaction startup and the magnitude of the hotspot was recorded to be ~259° C. However, with time, the bed temperature started to drop below the furnace set point and stabilized at a constant value. To counter the drop in the bed temperature and to maintain the temperature >190° C., the furnace temperature was increased to 240° C. Finally, the HDO product was collected and analyzed. A representative FID chromatogram is shown in FIG. 23. It can be observed that ~95% of the product were BA molecules with a carbon number of >23 and ~5% were <BA23 forming due to C—C cleavage.

TABLE 14
Reaction conditions and process parameters for deoxygenation of RKunsat-28/33
hydrogenated intermediate feed in a 1″ OD reactor.

| Parameter | Value | Units | Comments |
|---|---|---|---|
| Reactor OD | 1 | in | 316 L SS Tubing |
| Reactor ID | 0.834 | in | |
| Active Packing Length | 14 | in | PRICAT Ni 62/15T 5mmx4mm cylindrical |
| Weight of catalyst (W_{cat}) | 66.4 | g | pellets mixed with similar size alumina |
| Bed Dilution | 50 | % | pellets |
| (Wt % of catalyst) | | | |
| Reactor pressure | 600 | psig | This pressure is provided by the reactor |
| | | | Equilibar BPR |
| Furnace temperature | 230 | | The setpoint was adjusted based on the |
| | | | observed bed temperature |
| Liquid flow rate (Q_{liq}) | 2 | ml/min | Liquid flowrate through the Eldex pump was |
| | | | gradually increased to control the exotherm |
| Mass flow of H₂ | 10 | g/h | Setpoint on the MFC |
| LWHSV | 0.5 | $\frac{{g}_{reac}}{h \cdot {g}_{cat}}$ | Estimated from mass flow of RKsat-28/33 and catalyst weight |
| GWHSV | 0.2 | $\frac{{g}_{{H}_{2}}}{h \cdot {g}_{cat}}$ | Estimated from mass flow of H₂ and catalyst weight |
| Liquid superficial velocity | 0.01 | cm/s | $Estimated by using the correlation {U}_{s}^{L i q} =$ |
| $( {U}_{s}^{L i q} )$ | | | $\frac{{Q}_{liq}}{C . S . A . of the reactor}$ |
| Liquid mean residence time | 18.8 | min | $Estimated by using the correlation τ = \frac{ε {V}_{b e d}}{{Q}_{liq}}$ |
| | | | at various liquid flowrates while assuming a |
| | | | bed voidage (ε) of 0.3 |

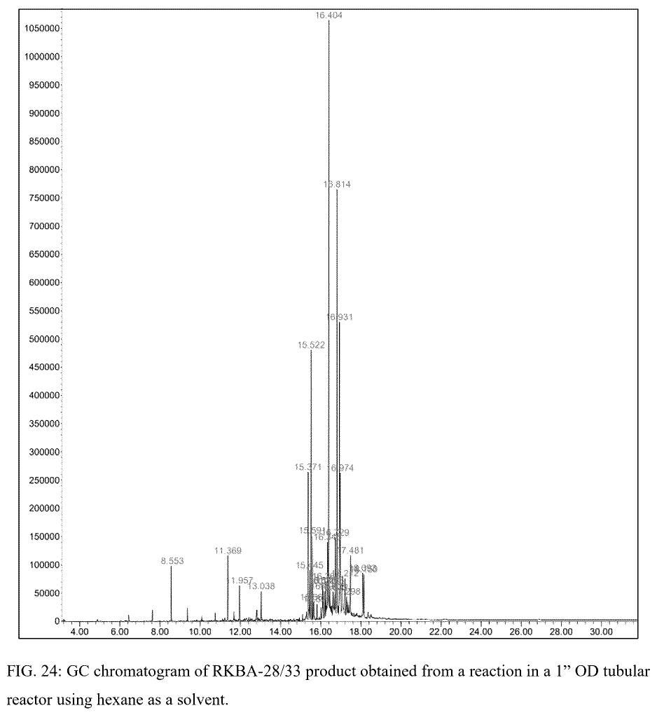

Example 8. Hydrodeoxygenation of RKunsat-28/33 of RKBA-28/33 in the flow reactor using hexane as the solvent. The hydrodeoxygenation reaction in the flow reactor, similar to Example 7, was performed by diluting RKunsat-28/33 in hexane instead of cyclohexane. The experimental protocol, and reaction conditions are similar to that shown in example 7 and Table 14. The GC analysis of the HDO product showed that ~95% of the product were BA molecules with a carbon number of >23 and ~5% were <BA23 forming due to C—C cleavage. A representative FID chromatogram is shown in FIG. 24.

### Example 9. Hydrodeoxygenation of RKunsat-14 of RKBA-14

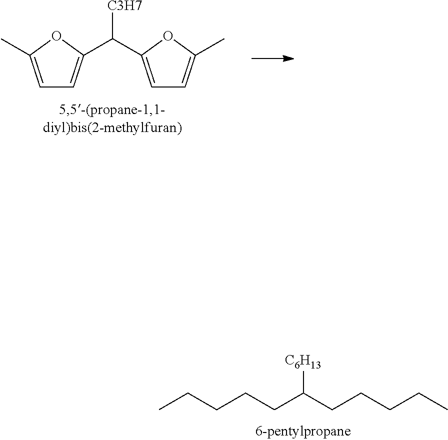

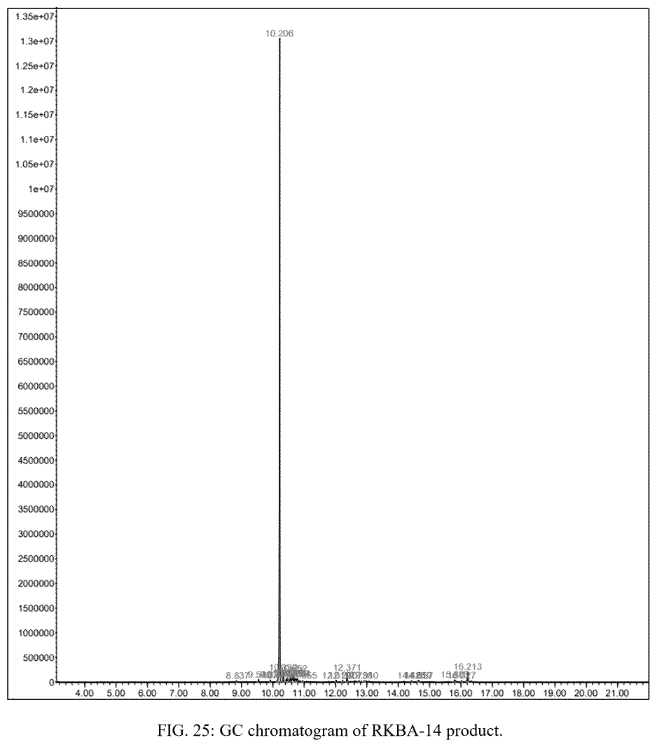

The HDO reaction RKunsat-14 to produce RKBA-14 was performed in a 2 L Parr reactor in the presence of a commercial Ni-based catalyst, Ni62/15P. An internal cooling coil was used in the reactor to control the reaction exotherm. The process conditions are shown in Table 15. To avoid reaction inhibition due to water by-product, intermittent water removal was performed after each segment. The total reaction time including catalyst activation was 15 hours. The GC chromatogram of the final HDO product is shown in FIG. 25. A conversion of 100% of RKunsat-14 and ~91% of RKBA-14 yield was achieved.

TABLE 15
Process parameters used for HDO of RKunsat-14
to BA14 in a Parr reactor.

| Entry | Step | Conditions |
|---|---|---|
| 1 | Activation and | 160 C. @ 5 bar H₂ for 2 hours |
| | hydrogenation | |
| 2 | Segment 1 | 240 C. @ 45 bar H₂ for 3 hours |
| 3 | Segment 2 | 240 C. @ 45 bar H₂ for 3 hours |
| 4 | Segment 3 | 240 C. @ 45 bar H₂ for 3 hours |
| 5 | Segment 4 | 240 C. @ 45 bar H₂ for 4 hours |

Example 10. Hydrogenation of RKunsat-15 of RKsatOH-15. Hydrogenation of RKunsat-15, a condensation product produced from 2MF and furfural by following similar reaction methodology as Example 1, was conducted in the 2 L Parr reactor in the presence of a commercial Ni-based catalyst, Ni62/15P at conditions of 5 bar H₂ and 160 C for 4 hours. An internal cooling coil was used in the reactor to control the reaction exotherm. GC analysis indicated formation of furan ring opened product (RKSatOH-15) with similar percentage of yield as tabulated in Table 7.

Specifications of Product. Key specifications of RKBA-14, RKBA17, RKBA-22 and RKBA28/33 products were determined by various ASTM methods. These results are summarized in Table 16.

TABLE 16
Specifications of Upcycled oils.

| | Boiling | Flash | | | | | | Freezing | Bio- | | Pour |
|---|---|---|---|---|---|---|---|---|---|---|---|
| | point | point | | Viscosity | Viscosity | | Viscosity | point | based | 12 | Point |
| Product | (° C.) | (° C.) | Density | at 40° C. | at 20° C. | RI | at 100° C. | (° C.) | carbon | number | (° C.) |
| RKBA- | 319 ± 5 | 153 ± 8° | 0.8049 | 6.31 cSt | 11.35 cSt | 1.4 - | 1.96 cSt | −10.5 ± −4 | 100% | — | −21 |
| 22 | | C. | g/mL | | | 1.44 | | | | | |
| | | | at 25° C. | | | | | | | | |
| RKBA- | 288 ± 12 | 121 ± 13° | 0.7895 | 3.42 cSt | 5.79 - | 1.4 | — | <− 40 ° C. | 100% | <0.3g/ | — |
| 17 | | C. | g/mL at | | 6.1 cSt | | | | | 100 g | |
| | | | 60º F | | | | | | | | |
| RKBA- | — | — | | 17.11 cSt | — | — | 3.808 | — | 100% | — | −48 |
| 28/33 | | | | | | | | | | | |
| RKBA- | — | 82.5 ± 9.5 | — | — | — | — | | — | — | — | — |
| 14 | | | | | | | | | | | |

The following ASTM methods were used for measuring specifications.
Density = ASTM D1209
Flash point = ASTM D93
Boiling point = ASTM D1120, ASTM D6352
Viscosity = ASTM D445
Freezing point = ASTM S2386
I2 number = USP-NF-2022, issue 1
Bio-based carbon = ASTM D6866-21 Method B (AMS)
Pour point = ASTM D6749

### Example 11. Cosmetic Products Formulation Using RKBA-17

### Example 11A. Cosmetic Composition for Bio-based Face Moisturizer

Water (65 wt. %), Zemea® (INCI name=propanediol; 10 wt. %) and Ronacare® magnesium sulfate (INCI name=magnesium sulfate; 1.0 wt. %) were pre-mixed to prepare Phase A. Similarly, RKBA-17 (20 wt. %) and Silube® 316 (INCI name=TMP lauryl dimethicone; 4.0 wt. %) were pre-mixed to prepare Phase B. Phase A was slowly added to Phase B until uniform and homogenize (cold mixing at room temperature). It provided a stable shade-based formulation for end-use bio-based facial moisturizer.

### Example 11B. Cosmetic Composition for Bio-based Super Soft Sunscreen Cream

Phase A was prepared by mixing water (42 wt. %), Zemea® (INCI name=propanediol; 10 wt. %), Spectrastat G2-N® (INCI name=Caprylhydroxamic Acid and) Glyceryl Caprylate (and) Glycerin; 1 wt %), RonaCare Magnesium Sulfate® (INCI name=Magnesium Sulfate; 0.5 wt %), and Biotiv L-Arginine (INCI name=Arginine; 0.5 wt %). Separately Phase B was prepared by mixing RKBA-17 (15 wt %), Zinclear XP65COCO® (INCI name=Zinc Oxide (and) Coco-Caprylate/Caprate (and) Polyglyceryl-3 Polyricinoleate (and) Isostearic Acid; 25 wt %), Silube316® (INCI name=TMP Lauryl Dimethicone; 3 wt %), SilwaxB116® (INCI name=Cetyl Dimethicone; 1 wt %) and SOLESPHERE H-53® (INCI name=Hydrated Silica; 2 wt %). Phase A was slowly mixed with Phase B with fast propeller stirring and homogenize briefly until glossy. This formulation provided white, glossy, super soft sunscreen cream for everyday facial use and moderate sun protection on the body. The formulation featured wonderful miscibility, stability, and aesthetics of the final product and minimum rubbing time needed to work out the whiteness of the zinc oxide active.

### Example 11C. Cosmetic Composition for Bio-based Eye Serum

Phase A was water (69.5 wt. %). Phase B was prepared by pre-mixing Zemea® (INCI name=propanediol; 10 wt. %) and Siligel® (INCI name=Acrylates Copolymer; 1.5 wt. %). Similarly, RKBA-17 (15 wt. %) and PolySuga® Mulse D6® (INCI name=Sorbitan Oleate Decylglucoside Crosspolymer; 4.0 wt. %) were pre-mixed to prepare Phase C. Phase B was slowly added to Phase

A with propeller stirring until uniform (Phase AB). Phase C was added to Phase AB with fast sharp-bladed impeller stirring and briefly homogenize until glossy. The formulation featured 100% bio-based eye serum using RKBA-17 that help make smooth homogenous solution with increased glossiness to keep under-eyes moisturized and bright.

### Example 11D. Cosmetic Composition for Bio-based Tinted Face Sun Protection Moisturizer

Phase A was prepared by pre-mixing water (39.75 wt. %), Zemea® (INCI name=propanediol; 10 wt. %) and RonaCare Magnesium Sulfate® (INCI name=Magnesium Sulfate; 0.5 wt %). Pbase B was prepared by pre-mixing Solaveil XT-300 (INCI name=Titanium Dioxide (and) Caprylic/Capric Triglyceride (and) Polhydroxystearic Acid (and) Stearic Acid (and) Alumina; 30 wt %), RKBA-17 (10 wt %); Silube316® (INCI name=TMP Lauryl Dimethicone; 2 wt %), Span 80 (INCI name=Sorbitan Oleate; 2 wt %) and SOLESPHERE H-53® (INCI name=Hydrated Silica; 2 wt %). Separately, Phase C was prepared by pre-mixing yellow iron oxide (INCI name=iron oxides; 3.25 wt %), red iron oxide (INCI name=iron oxides; 0.4 wt %) and black iron oxide (INCI name=iron oxides; 0.1 wt %). Phase A was slowly added to Phase B with fast dispersion blade stirring until uniform to obtain Phase AB. Phase C was added to Phase AB with dispersion blade until color is uniform.

### Example 12. Lubricant Products Formulation Using RKBA-22

RKBA-12 oil was formulated with ISO68 oil and pour point depressant (PPD) to improve viscosity and pour points of the formulated products for turbine oil applications.

### Example 12A. Formulation with ISO68 Oil

Formulation of RKBA-22 oil with different amounts of ISO68 oil and the specifications of the formulated oils are shown in Table 17. Viscosity at 40° C. (KV40) and 100° C. (KV100) of the formulated products were determined using an Anton Paar SVM 3000/G2 Stabinger Viscometer. Viscosity Index (VI) values were calculated using standard equations. Pour points were determined by ASTM D5949 method.

TABLE 17
Formulation recipes of RKBA-22 oil with ISO68 oil
and specifications of the formulated oils.

| | RKBA- | ISO68 | KV40 | KV100 | | | Pour |
|---|---|---|---|---|---|---|---|
| Entry | 22 (g) | (g) | (cSt) | (cSt) | VI | Density | point |
| A | 4.00 | 1.00 | 11.3 | 2.88 | 102 | 0.828 | |
| B | 3.00 | 2.00 | 15.9 | 3.64 | 112 | 0.838 | |
| C | 2.50 | 2.50 | 19.3 | 4.10 | 113 | 0.843 | |
| D | 2.00 | 3.00 | 23.9 | 4.90 | 115 | 0.848 | |
| E | 1.50 | 3.50 | 30.4 | 5.41 | 112 | 0.853 | |
| F | 1.00 | 4.00 | 39.2 | 6.37 | 111 | 0.858 | |
| G | 1.40 | 3.60 | 31.8 | 5.59 | 114 | 0.801 | (−) 15 |
| H | 1.30 | 3.70 | 33.6 | 5.81 | 115 | 0.802 | |
| K | 1.20 | 3.80 | 35.5 | 6.02 | 114 | 0.803 | |
| L | 1.10 | 3.90 | 37.3 | 6.19 | 113 | 0.804 | |
| M | 0.90 | 4.10 | 40.8 | 6.57 | 113 | 0.806 | |
| N | 0.80 | 4.20 | 43.7 | 6.78 | 110 | 0.807 | |
| O | 0.70 | 4.30 | 45.7 | 7.02 | 111 | 0.808 | (−) 12 |
| P | 0.60 | 4.40 | 48.4 | 7.26 | 110 | 0.810 | |
| Q | 0.50 | 4.50 | 51.1 | 7.49 | 108 | 0.811 | |

### Example 12B. Formulation with ISO68 Oil

Two formulated oils of Table 17 having KV40 value of 31.8 and 45.7 were further formulated with three different PPD of three different treat rates. Pour point and cloud points of the formulated oils were determined by ASTM D5949 and ASTM D5773 methods, respectively. The results are shown in Table 18.

TABLE 18
Pour points and cloud points of neat RKBA-22 and formulated oils of VI 31.8 and 45.7
of Table 17.

| Pour point depressant | | Viscoplex 1-154 | | | Viscoplex 1-850 | | | Viscoplex 1-180 | | |
|---|---|---|---|---|---|---|---|---|---|---|
| Treat rate (%) | | 0.2% | 0.5% | 1% | 0.2% | 0.5% | 1% | 0.2% | 0.5% | 1% |
| Formulation of | Pour point | −18 | −18 | −18 | −15 | −18 | −18 | −18 | −18 | −18 |
| neat RKBA-22 | Cloud point | −14.3 | −14.9 | −15.6 | −13.9 | −14.3 | −15.2 | −14.7 | −15.0 | −15.7 |
| | (° C.) | | | | | | | | | |
| Formulation of | Pour point | −21.0 | −21.0 | −18.0 | −18.0 | −18.0 | −18.0 | −18.0 | | |
| oil of VI 31.8 | (°C) | | | | | | | | | |
| from Table 17 | Cloud point | −10.8 | −11.7 | −12.3 | −12.2 | −12.8 | −10.6 | −10.6 | | |
| | (° C.) | | | | | | | | | |
| Formulation of | Pour point | −24.0 | −24.0 | −21.0 | −21.0 | −24.0 | −24.0 | | | |
| oil of VI 45.7 oil | (° C.) | | | | | | | | | |
| from Table 17 | Cloud point | −12.1 | −12.9 | −12.7 | −13.5 | −11.3 | −11.4 | | | |
| | (°° C.) | | | | | | | | | |

1. A compound having the following structure: 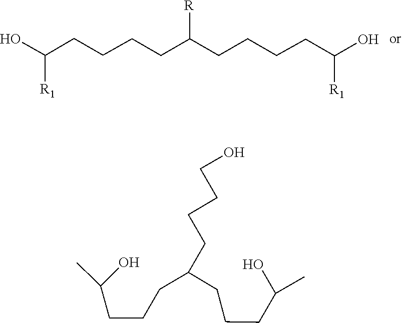
wherein R is methyl, npropyl, nhexyl, nbutyl, n-pentyl, or nundecyl and R₁ is independently selected from H or an alkyl chain of carbon number 1-7.

2. A composition comprising the compound of claim 1, wherein the compound has at least one branched carbon chain; the composition comprises a bio-based content in the range of 20 to 100%, according to ASTM-D6866 and hydroxyl content of greater than 20 mg KOH/per gram, according to ASTM 4274-99 method.

3. A method of making compounds having the following structures: 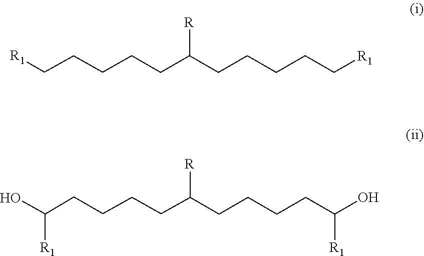
wherein R is methyl, npropyl, nhexyl, nbutyl, n-pentyl, or nundecyl and R₁ is independently selected from H or an alkyl chain of carbon number 1-7
wherein the method comprises of the steps of:
(a) an acid catalyzed condensation reaction of a first component comprising one of more of a 2-alkylfuran wherein the alkyl group is independently selected from carbon number of 1 to 7, with a second component comprising an aldehyde having the formula of O═CH—R wherein R is methyl, npropyl, nhexyl, nbutyl, n-pentyl, or nundecyl, to produce a condensation compound,
(b) optionally selective hydrogenation of the condensation compound in the presence of a hydrogenation catalyst to obtain a hydrogenated saturated or ring-opened hydroxyl compound,
(c) selective hydrodeoxygenation of the condensation compound or the hydrogenated saturated or ring-opened hydroxyl compound, in the presence of a hydrodeoxygenation catalyst to obtain a branched alkane compound.

4. A method of making compounds having the structure: 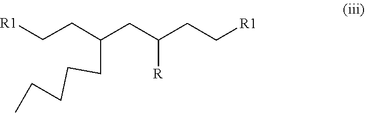
wherein R1 is independently selected from alkyl groups of carbon number 2 to 14, R is independently selected from H or —(CH2)4 group,
wherein the method comprises of the steps of:
(a) a base catalyzed condensation reaction of a third component comprising one of more of a ketone of molecular formulae (R1)(R1) C═O wherein R1 is independently selected from alkyl groups of carbon number from 2 to 14, with furfural to produce compounds of structure (i): 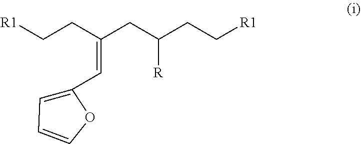
wherein R is H or a furan ring,
(b) optionally selective hydrogenation of the condensation compound in the presence of a hydrogenation catalyst to obtain furan rings saturated intermediate or ring-opened hydroxyl product to produce compounds of structure (ii): 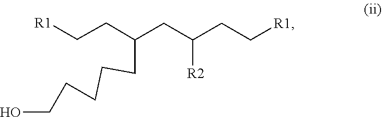
wherein and R2 is H or —(CH2)3CH₂OH group
(c) selective hydrodeoxygenation of the condensation compound of structure (i) or the hydrogenated saturated or ring-opened hydroxyl intermediate of structure (ii) in the presence of a hydrodeoxygenation catalyst to obtain a branched alkane compound of structure (iii).

5. The method of claim 3, wherein the acid catalyst comprises one acidic catalyst selected from the group consisting of acidic resins, fluorinated resins, zeolites, phosphoric acid, phosphorous silica, orthophosphoric acid, HCl, H2SO4, methanesulfonic acid, and p-tolunesulfonic acid.

6. The method of claim 4, wherein the base catalyst comprises one base catalyst selected from the group consisting of CaO, MgO, Al—Mg hydrotalcites, NaOH, KOH, Ca(OH)2, and Mg(OH)2.

7. The method of claim 3, wherein the hydrogenation catalyst comprises one metal catalyst selected from the group consisting of Ni, Pd, Pt, Ru, supported on a support material selected from activated carbon, porous carbon, polymeric hybrid material, or weakly acidic materials, and mixed Ni catalyst in the presence of NiO, Al2O3, SiO2, Cr2O3, ZrO2, or Kieselguhr.

8. The method of claim 3, wherein the hydrogenation reaction is conducted in a metallic pressure reactor or in a flow reactor under H₂ pressure ranging from 1 bar to 45 bar and temperature in the range of 40° C. to 200° C.

9. The method claim 3, wherein the hydrodeoxygenation catalyst comprises a bifunctional catalyst in powder and pellet forms selected from Ni/ZSM5, Ni/zeolite, Ni/silica, Ni/Al2O3, Pd/ZSM5, Pd/zeolite, Pd/silica, Pd/Al2O3, a physically mixed oxide catalysts consisting of at least one metal oxide and at least one acidic oxide, or a supported metal-metal oxide catalyst of a formula M¹MO wherein M¹=Ir, Ru, Ni, Co, Pd, or Rh and M=Re, Mo, W, Nb, Mn, V, Ce, Cr, Zn, Co, Y, or Al.

10. The method of claim 3, wherein the hydrodeoxygenation reactions are conducted in the flow reactor comprising a catalyst bed and temperature ranging from 150° C. to 300° C. under varying flow rates of feed and H2 pressure ranging from 80 psi to 800 psi.

11. A composition comprising a compound made by the method of claim 3, wherein the composition has a bio-based carbon content in the range of 10% to 100%, according to ASTM-D6866.

12. A compound made by the method of claim 3, wherein the compound has at least one branched carbon chain.

13. A cosmetic product formulation comprising 0.1-99% by weight of one or more compound of claim 1 as an emollient and an effective amount of one or more cosmetic additives.

14. The cosmetic product formulation of claim 13 further comprising a compound of structure of: 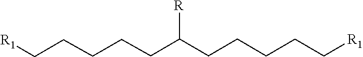
wherein R is methyl, npropyl, nhexyl, nbutyl, n-pentyl, or nundecyl and R₁ is independently selected from H or an alkyl chain of carbon number 1-7; or 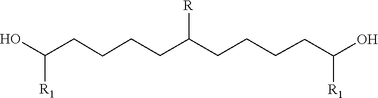
wherein R is methyl, npropyl, nhexyl, nbutyl, n-pentyl, or nundecyl and R₁ is independently selected from H or an alkyl chain of carbon number 1-7.

15. The cosmetic product formulation of claim 13 further comprising a cosmetic oil.

16. A lubricant product formulation comprising 0.1-99% by weight of one or more compound of claim 1 and an effective amount of one or more lubricant additives.

17. The lubricant product formulation of claim 16 further comprising a compound of structure of: 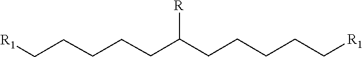
wherein R is methyl, npropyl, nhexyl, nbutyl, n-pentyl, or nundecyl and R₁ is independently selected from H or an alkyl chain of carbon number 1-7; 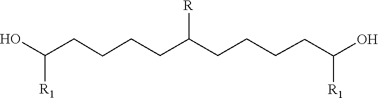
wherein R is methyl, npropyl, nhexyl, nbutyl, n-pentyl, or nundecyl and R₁ is independently selected from H or an alkyl chain of carbon number 1-7; or 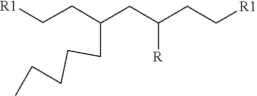
wherein R1 is independently selected from alkyl groups of carbon number 2 to 14, R is independently selected from H or —(CH2)4 group.

18. A cosmetic product composition comprising
(i) emollients comprising one or more compounds of claim 1, and
(ii) one or more additives selected from the group of pigment, fragrance, emulsifier, wetting agent, thickener, emollient, rheology modifier, viscosity modifier, gelling agent, antiperspirant agent, deodorant active, fatty acid salt, film former, anti-oxidant, humectant, opacifier, monohydric alcohol, polyhydric alcohol, fatty alcohol, preservative, pH modifier, a moisturizer, skin conditioner, stabilizing agent, proteins, skin lightening agents, topical exfoliants, antioxidants, retinoids, refractive index enhancer, photo-stability enhancer, SPF improver, UV blocker, antibiotic agents, antiseptic agents, antifungal agents, corticosteroid agents, anti-acne agents, and water.

19. A lubricant composition comprising
(i) a base oil comprising one or more compounds of claim 1, and
(ii) a lubricant additive selected from thickener, viscosity modifier, base oils, viscosity index improver, pour point depressant, anti-wearing agent, corrosion inhibitor, anti-oxidant, extreme pressure additive, detergents, and anti-foaming agent.

20. A compound comprising the structure of: 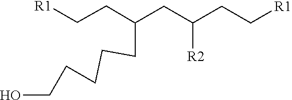
wherein R1 is independently selected from alkyl groups of carbon number 2 to 14, and R2 is H or —(CH2)4.

21. A polymer product synthesis using 1-90 wt % of one or more compounds of claim 1.

22. The polymer product according to claim 21 wherein at least one or more compound has a fully saturated furan ring opened moiety or a partially saturated furan-ring opened moiety containing a hydroxyl group of structure RKsatOH-n.
